(19) United States
(12) Patent Application Publication
(10) Pub. No.: US 2021/0292722 A1
(43) Pub. Date: Sep. 23, 2021

(54) NOVEL CRISPR-ASSOCIATED PROTEIN AND USE THEREOF

(71) Applicants: G+FLAS LIFE SCIENCES, Seoul (KR); SEOUL NATIONAL UNIVERSITY R&DB FOUNDATION, Seoul (KR)

(72) Inventors: Sunghwa CHOE, Seoul (KR); Han Seong KIM, Seoul (KR); Dong Wook KIM, Seoul (KR); Jongjin PARK, Seoul (KR); Jiyoung YOON, Seoul (KR)

(73) Assignees: G+FLAS LIFE SCIENCES, Seoul (KR); SEOUL NATIONAL UNIVERSITY R&DB FOUNDATION, Seoul (KR)

(21) Appl. No.: 17/266,882

(22) PCT Filed: Aug. 9, 2019
(86) PCT No.: PCT/KR2019/010110

§ 371 Date: Feb. 8, 2021

(30) Foreign Application Priority Data
Aug. 9, 2018 (KR) 10-2018-0093336

(51) Int. Cl. C12N 9/22; A61K 38/46; C12N 15/11; A61K 31/7088

(52) U.S. Cl. CPC C12N 9/22; A61K 38/465; C12N 2310/20; A61K 31/7088; C12N 2800/80; C12N 15/11

(57) ABSTRACT

A novel CRISPR-associated protein and a use thereof are disclosed. A protein of the amino acid sequence of SEQ ID NO: 1 or SEQ ID NO: 3 exhibits the activity of endonucleases, which recognize and cleave an intracellular nucleic acid sequence linked to a guide RNA. Therefore, a novel CRISPR-associated protein can be used as a different nuclease for genome editing, in a CRISPR-Cas system.

### TECHNICAL FIELD

The present invention relates to a novel CRISPR-associated protein and a use thereof.

### BACKGROUND ART

Genome editing is a technique by which the genetic information of a living organism is freely edited. Advances in the field of life sciences and development in genome sequencing technology have made it possible to understand a wide range of genetic information. For example, understanding of genes for reproduction of animals and plants, diseases and growth, genetic mutations that cause various human genetic diseases, and production of biofuels has already been achieved; however, further technological advances must be made to directly utilize this understanding for the purpose of improving living organisms and treating human diseases.

Genome editing techniques can be used to change the genetic information of animals, including humans, plants, and microorganisms, and thus their application range can be dramatically expanded. Genetic scissors, which are molecular tools designed and made to precisely cut desired genetic information, play a key role in genome editing techniques. Similar to the next-generation sequencing techniques that took the field of gene sequencing to the next level, use of the gene scissors is becoming a key technique in increasing the speed and range of utilization of genetic information and creating new industrial fields.

The genetic scissors having been developed so far may be divided into three generations according to the order of their appearance. The first generation of genetic scissors is zinc finger nuclease (ZFN); the second generation of genetic scissors is transcription activator-like effector nuclease (TALEN); and the most recently studied, clustered regularly interspaced short palindromic repeat (CRISPR)/CRISPR-associated protein 9 (Cas9) is the third generation of genetic scissors.

The CRISPRs are loci containing multiple short direct repeats that are found in the genomes of approximately 40% of sequenced bacteria and 90% of sequenced archaea. The Cas9 protein forms an active endonuclease when complexed with two RNAs termed CRISPR RNA (crRNA) and trans-activating crRNA (tracrRNA), thereby slicing foreign genetic elements in invading phages or plasmids to protect the host cells. The crRNA is transcribed from the CRISPR element of the host genome that has previously been occupied by foreign invaders.

RNA-guided nucleases derived from this CRISPR-Cas system provide a tool capable of genome editing. In particular, studies have been actively conducted which are related to techniques capable of editing genomes of cells and organs using a single-guide RNA (sgRNA) and a Cas protein. Recently, Cpf1 protein (derived from Prevotella and Francisella 1) was reported as another nuclease protein in the CRISPR-Cas system (B. Zetsche, et al., 2015), which results in a wider range of options in genome editing.

### DISCLOSURE OF INVENTION

### Technical Problem

As a result of making continuous efforts to develop a protein that is more effective in genome editing than the known nucleases, the present inventors have found a novel CRISPR-associated protein that recognizes and cleaves a target nucleic acid sequence, and thus have completed the present invention.

Accordingly, an object of the present invention is to provide a novel CRISPR-associated protein that recognizes and cleaves a target nucleic acid sequence.

### Solution to Problem

To achieve the above-mentioned object, the present invention provides a Cas12a protein having the amino acid sequence of SEQ ID NO: 1.

In addition, the present invention provides a Cas12a protein having the amino acid sequence of SEQ ID NO: 1, of which lysine (Lys) at position 925 is substituted with another amino acid.

In addition, the present invention provides a Cas12a protein having the amino acid sequence of SEQ ID NO: 3.

In addition, the present invention provides a Cas12a protein having the amino acid sequence of SEQ ID NO: 3, of which lysine (Lys) at position 930 is substituted with another amino acid.

In addition, the present invention provides a Cas12a protein having the amino acid sequence of SEQ ID NO: 1, of which aspartic acid (Asp) at position 877 is substituted with another amino acid.

In addition, the present invention provides a Cas12a protein having the amino acid sequence of SEQ ID NO: 3, of which aspartic acid (Asp) at position 873 is substituted with another amino acid.

In addition, the present invention provides a pharmaceutical composition for treating cancer, comprising as active ingredients: mgCas12a; and crRNA that targets a nucleic acid sequence specifically present in cancer cells.

### Advantageous Effects of Invention

The protein represented by the amino acid sequence of SEQ ID NO: 1 or SEQ ID NO: 3, according to the present invention, has endonuclease activity that recognizes and cleaves an intracellular nucleic acid sequence bound to a guide RNA. Therefore, the novel CRISPR-associated protein of the present invention can be used as another nuclease, which performs genome editing, in the CRISPR-Cas system.

### BRIEF DESCRIPTION OF DRAWINGS

FIG. 1 illustrates a schematic diagram of a process of discovering Cas12a from metagenome.

FIG. 2A illustrates a phylogenetic tree of the discovered Cas12a.

FIG. 2B illustrates structures of novel Cas12a's and AsCas12a.

FIGS. 3 to 8 illustrate amino acid sequences of existing Cas12a's and the mgCas12a's of the present invention, which have been aligned using the ESPript program.

FIGS. 9A and 9B illustrate tables obtained by comparing and summarizing the sequence information of the Cas12a's and the mgCas12a's of the present invention.

FIGS. 10 to 12 illustrate results obtained by identifying activity, depending on pH, of the mgCas12a's according to the present invention. On the other hand, crRNA #1 in FIG. 10 has the nucleotide sequence of SEQ ID NO: 25, and crRNA #2 in FIG. 11 has the nucleotide sequence of SEQ ID NO: 26.

FIG. 13 illustrates a diagram in which a target nucleic acid sequence and positions where crRNAs bind are indicated.

FIG. 14 illustrates results obtained by identifying gene editing efficiency achieved by respective proteins (mock, mgCas12a-1, and mgCas12a-2) in a case where crRNA for each of the genes CCR5 and DNMT1 is used.

FIG. 15 illustrates results obtained by identifying gene editing efficiency achieved by respective proteins (FnCpf1, mgCas12a-1, and mgCas12a-2) in a case where two crRNAs for the respective genes FucT14-1 and FucT14-2 are used.

FIGS. 16A and 16B illustrates results obtained by identifying DNA cleavage activity of FnCas12a, WT mgCas12a-1, or WT mgCas12a-2 protein.

FIG. 17 illustrates results obtained by identifying non-specific DNase functions of existing Cas12a (AsCas12a, FnCas12a, or LbCas12a) and novel Cas12a (WT mgCas12a-1, d_mgCas12a-1, WTmgCas12a-2, or d_mgCas12a-2).

FIGS. 18A and 18B illustrate results obtained by identifying whether the FnCas12a, WT mgCas12a-1, or WT mgCas12a-2 protein has a non-specific DNase function without crRNA.

FIG. 19 illustrates results obtained by identifying whether the mgCas12a can perform DNA cleavage using 5′ handle of existing Cas12a.

FIGS. 20A and 20B illustrate DNA cleavage activity of the FnCas12a, mgCas12a-1, or mgCas12a-2 protein in divalent ions.

### BEST MODE FOR CARRYING OUT THE INVENTION

In an aspect of the present invention, there is provided a novel Cas12a protein obtained from metagenome.

As used herein, the term “Cas12a” is a CRISPR-related protein and may also be referred to as Cpf1. In addition, Cpf1 is an effector protein found in type V CRISPR systems. Cas12a, which is a single effector protein, is similar to Cas9, which is an effector protein found in type II CRISPR systems, in that it combines with crRNA to cleave a target gene. However, the two differ in how they work. The Cas12a protein works with a single crRNA. Therefore, for the Cas12a protein, there is no need to simultaneously use crRNA and trans-activating crRNA (tracrRNA) or to create a single-guide RNA (sgRNA) by synthetic combination of tracrRNA and crRNA, as in Cas9.

In addition, unlike Cas9, the Cas12a system recognizes a PAM present at the 5′ position of a target sequence. In addition, in the Cas12a system, a guide RNA that determines a target also has a shorter length than Cas9. In addition, Cas12a is advantageous in that it generates a 5′ overhang (sticky end), rather than a blunt end, at a cleavage site in a target DNA, and thus enables more accurate and diverse gene editing.

Conventionally, the Cas12a proteins may be derived from the Candidatus genus, the Lachnospira genus, the Butyrivibrio genus, the Peregrinibacteria genus, the Acidominococcus genus, the Porphyromonas genus, the Prevotella genus, the Francisella genus, the Candidatus Methanoplasma genus, or the Eubacterium genus. Specifically, PbCas12a is a protein derived from Parcubacteria bacterium GWC2011_GWC2_44_17; PeCas12a is a protein derived from Peregrinibacteria Bacterium GW2011_GWA_33_10; AsCas12a is a protein derived from Acidaminococcus sp. BVBLG; PmCas12a is a protein derived from Porphyromonas macacae; LbCas12a is a protein derived from Lachnospiraceae bacterium ND2006; PcCas12a is a protein derived from Porphyromonas crevioricanis; PdCas12a is a protein derived from Prevotella disiens; and FnCas12a is a protein derived from Francisella novicida U112. However, each Cas12a protein may have different activity depending on the microorganism from which it is derived.

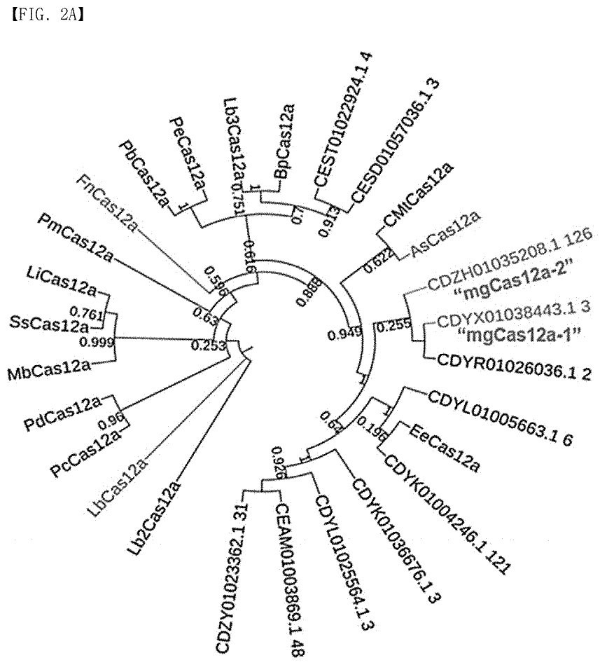

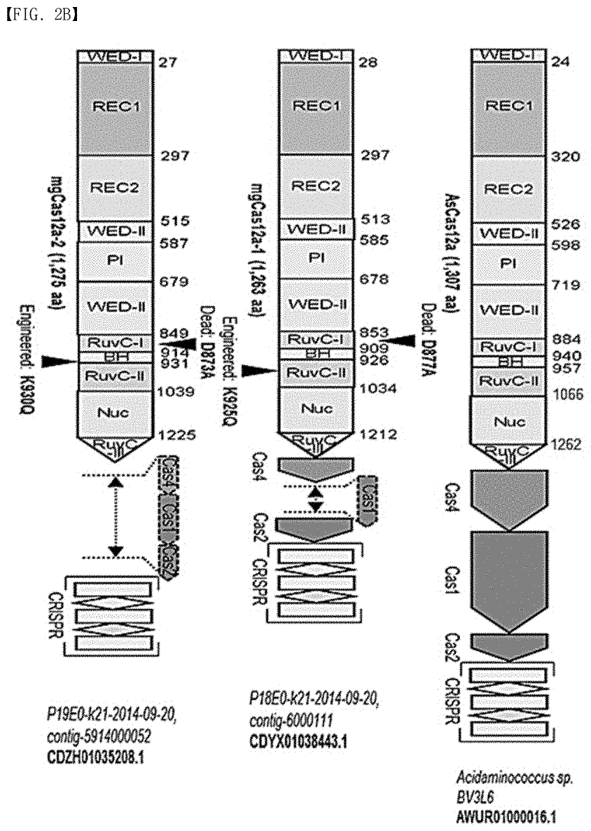

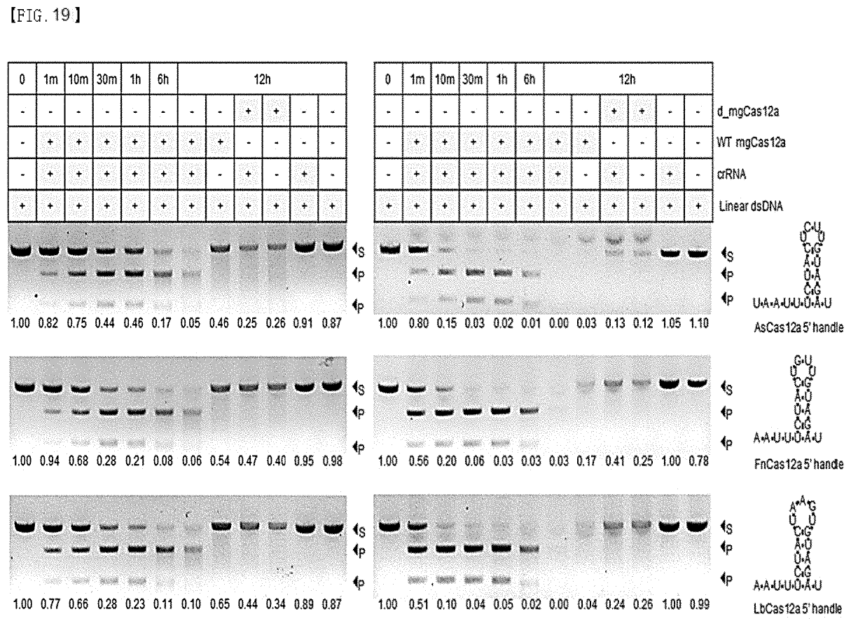

In the present invention, novel Cas12a's have been identified by analyzing genes in metagenomes. Hereinafter, metagenome-derived Cas12a may be referred to as mgCas12a. Like AsCas12a, the mgCas12a of the present invention includes WED, REC, PI, RuvC, BH, and NUC domains (FIG. 2). In addition, it was identified that similar to previously known Cas12a proteins, the mgCas12a protein of the present invention can perform gene cleavage with a gRNA including crRNA and 5′-handle. It was identified that the mgCas12a uses 5′-handle RNA having the same sequence as FnCas12a. Specifically, the 5′-handle RNA may have a sequence of AAUUUCUACUGUUGUAGAU (SEQ ID NO: 12). However, it was identified that the mgCas12a works even with a 5-handle RNA in AsCas12a and LbCas12a (FIG. 19).

The mgCas12a may additionally include a tag for separation and purification. The tag may be bound to the N-terminus or C-terminus of the mgCas12a. In addition, the tag may be bound simultaneously to the N-terminus and C-terminus of the mgCas12a. One specific example of the tag may be a 6×His tag.

As one specific example of the mgCas12a, there is provided a protein having the amino acid sequence of SEQ ID NO: 1. In addition, as long as activity of the mgCas12a is not changed, deletion or substitution of part of the amino acids may be made therein. Specifically, the mgCas12a may be a protein having the amino acid sequence of SEQ ID NO: 1, of which lysine (Lys) at position 925 is substituted with another amino acid. Here, the other amino acid may be any one selected from the group consisting of arginine (Arg), histidine (His), aspartic acid (Asp), glutamic acid (Glu), serine (Ser), threonine (Thr), asparagine (Asn), glutamine (Gln), tyrosine (Tyr), alanine (Ala), isoleucine (Ile), leucine (Leu), valine (Val), phenylalanine (Phe), methionine (Met), tryptophan (Trp), glycine (Gly), proline (Pro), and cysteine (Cys). Specifically, the protein may have the amino acid sequence of SEQ ID NO: 1, of which lysine at position 925 is substituted with glutamine. That is, the protein may have the amino acid sequence of SEQ II) NO: 5.

In addition, the gene that encodes the protein having the amino acid sequence of SEQ ID NO: 1 may be a polynucleotide having the nucleotide sequence of SEQ ID NO: 2. In addition, the mgCas12a having the amino acid sequence of SEQ ID NO: 1, according to the present invention, may have optimal activity at pH 7.0 to pH 7.9.

As another specific example of the mgCpf1, there is provided a protein having the amino acid sequence of SEQ ID NO: 3. In addition, as long as activity of the mgCpf1 is not changed, deletion or substitution of part of the amino acids may be made therein. Specifically, the mgCpf1 may be a protein having the amino acid sequence of SEQ ID NO: 3, of which lysine (Lys) at position 930 is substituted with another amino acid. Here, the other amino acid may be any one selected from the group consisting of arginine (Arg), histidine (His), aspartic acid (Asp), glutamic acid (Glu), serine (Ser), threonine (Thr), asparagine (Asn), glutamine (Gln), tyrosine (Tyr), alanine (Ala), isoleucine (Ile), leucine (Leu), valine (Val), phenylalanine (Phe), methionine (Met), tryptophan (Trp), glycine (Gly), proline (Pro), and cysteine (Cys). Specifically, the protein may have the amino acid sequence of SEQ ID NO: 3, of which lysine at position 930 is substituted with glutamine. That is, the protein may have the amino acid sequence of SEQ ID NO: 6.

The gene that encodes the protein having the amino acid sequence of SEQ ID NO: 3 may be a polynucleotide having the nucleotide sequence of SEQ ID NO: 4.

In addition, the mgCas12a having the amino acid sequence of SEQ ID NO: 3, according to the present invention, may have optimal activity at pH 7.0 to pH 7.9.

In another aspect of the present invention, there is provided an mgCas12a protein with decreased endonuclease activity. One specific example thereof may be mgCas12a having the amino acid sequence of SEQ ID NO: 1, of which aspartic acid (Asp) at position 877 is substituted with another amino acid. Here, the other amino acid may be any one selected from the group consisting of arginine (Arg), histidine (His), glutamic acid (Glu), serine (Ser), threonine (Thr), asparagine (Asn), glutamine (Gln), tyrosine (Tyr), alanine (Ala), lysine (Lys), isoleucine (Ile), leucine (Leu), valine (Val), phenylalanine (Phe), methionine (Met), tryptophan (Trp), glycine (Gly), proline (Pro), and cysteine (Cys). Specifically, the protein may be a protein obtained by substitution of the aspartic acid (Asp) with alanine (Ala).

Another specific example of the mgCas12a protein may be mgCas12a having the amino acid sequence of SEQ ID NO: 3, of which aspartic acid (Asp) at position 873 is substituted with another amino acid. Here, the other amino acid may be any one selected from the group consisting of arginine (Arg), histidine (His), glutamic acid (Glu), serine (Ser), threonine (Thr), asparagine (Asn), glutamine (Gln), tyrosine (Tyr), alanine (Ala), lysine (Lys), isoleucine (Ile), leucine (Leu), valine (Val), phenylalanine (Phe), methionine (Met), tryptophan (Trp), glycine (Gly), proline (Pro), and cysteine (Cys). Specifically, the protein may be a protein obtained by substitution of the aspartic acid (Asp) with alanine (Ala). Here, the mgCas12a with decreased endonuclease activity may be referred to as dead mgCas12a or d_mgCas12a. The d_mgCas12a may have the amino acid sequence of SEQ ID NO: 13 or SEQ ID NO: 14.

In addition, in yet another aspect of the present invention, there is provided a pharmaceutical composition for treating cancer, comprising as active ingredients: mgCas12a; and crRNA that targets a nucleic acid sequence specifically present in cancer cells. Here, the mgCas12a may have any one amino acid sequence selected from the group consisting of SEQ ID NO: 1, SEQ ID NO: 3, SEQ ID NO: 5, and SEQ ID NO: 6. As used herein, the term “nucleic acid sequence specifically present in cancer cells” refers to a nucleic acid sequence that is not present in normal cells and is present only in cancer cells. That is, this term refers to a sequence different from that in normal cells, and the two sequences may differ by at least one nucleic acid. In addition, such a difference may be caused by substitution or deletion of part of the gene. As one specific example, the nucleic acid sequence specifically present in cancer cells may be an SNP present in cancer cells. A target DNA having the above-mentioned sequence, which is present in cancer cells, and a guide RNA having a sequence complementary to the target DNA specifically bind to each other.

In particular, regarding the nucleic acid sequence specifically present in cancer cells, crRNAs can be created by finding specific SNPs, which exist only in cancer cells, through genome sequencing of various cancer tissues and using the same. This is done in a way of exhibiting cancer cell-specific toxicity, and thus makes it possible to develop patient-specific anti-cancer therapeutic drugs. In addition, the nucleic acid sequence specifically present in cancer cells may be a gene having high copy number variation (CNV) in cancer cells, unlike normal cells.

One specific example of the cancer may be any one selected from the group consisting of bladder cancer, bone cancer, blood cancer, breast cancer, melanoma, thyroid cancer, parathyroid cancer, bone marrow cancer, rectal cancer, throat cancer, laryngeal cancer, lung cancer, esophageal cancer, pancreatic cancer, gastric cancer, tongue cancer, skin cancer, brain tumor, uterine cancer, head or neck cancer, gallbladder cancer, oral cancer, colon cancer, perianal cancer, central nervous system tumor, liver cancer, and colorectal cancer. In particular, the cancer may be gastric cancer, colorectal cancer, liver cancer, lung cancer, and breast cancer, which are known as the five major cancers in Korea.

Here, crRNA that targets the nucleic acid sequence specifically present in cancer cells may include one or more gRNA sequences. For example, the crRNA may use a gRNA capable of simultaneously targeting exons 10 and 11 of BRCA1 present in ovarian cancer or breast cancer. In addition, the crRNA may use two or more gRNAs targeting exon 11 of BRCA1. As such, combination of gRNAs may be appropriately selected depending on purposes of cancer treatment and types of cancer. That is, different gRNAs may be selected and used.

### MODE FOR THE INVENTION

Hereinafter, the present invention will be described in more detail by way of the following examples. However, the following examples are for illustrative purposes only, and the scope of the present invention is not limited thereto.

### Example 1. Discovery of Metagenome-Derived Cas12a Protein

Metagenome nucleotide sequences were downloaded from the NCBI Genbank BLAST database and built into a local BLASTp database. In addition, 16 Cas12a's and various CRISPR-related protein (Cas1) amino acid sequences were downloaded from the Uniprot database. The MetaCRT program was used to find CRISPR repeats and spacer sequences in the metagenome. Then, only the metagenome sequences having the CRISPR sequence were extracted and their genes were predicted using the Prodigal program.

Among the predicted genes, those within a range that is 10 kb upstream or downstream of the CRISPR sequence were extracted, and the amino acid sequence of Cas12a was used to predict a Cas12a homolog among the genes in question. The Cas1 gene was used to predict whether there was a Cas1 homolog upstream or downstream of the Cas12a homolog; and Cas12a genes ranging from 800 aa to 1,500 aa, which had Cas1 around, were selected. For each of these genes, BLASTp was used in the NCBI Genbank non-redundant database to determine whether the gene was a gene that had already been reported or whether the gene was a gene having no association with CRISPR at all.

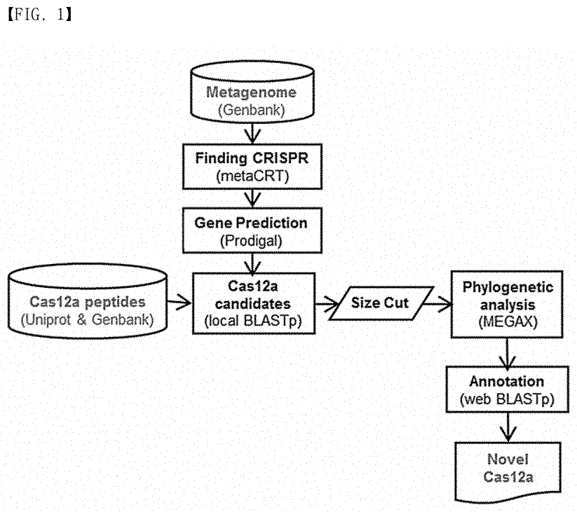

After removing fragmented Cas12a's that do not start with methionine (Met), these genes were aligned using a multiple alignment using fast fourier transform (MAFFT) program. Then, a phylogenetic tree was drawn with Neighbor-joining (100× bootstrap) using MEGA7. The gene that forms a monophyletic taxon with the previously known Cas12a gene was selected, and a phylogenetic tree thereof was drawn, together with the amino acid sequence of the existing Cas12a, using MEGA7, maximum-likelihood, and 1000× bootstrap, to examine their evolutionary relationship. Here, the process of discovering Cas12a from the metagenome is schematically illustrated in FIG. 1. In addition, the phylogenetic tree of the Cas12a is illustrated in FIG. 2A. Here, a novel protein having the amino acid sequence of SEQ ID NO: 1 was named WT mgCas12a-1. In addition, a novel protein having the amino acid sequence of SEQ ID NO: 3 was named WT mgCas12a-2. In addition, the structures of AsCas12a, mgCas12a-1, and mgCas12a-2 are illustrated in FIG. 2B.

### Example 2. Production of Variants of mgCas12a

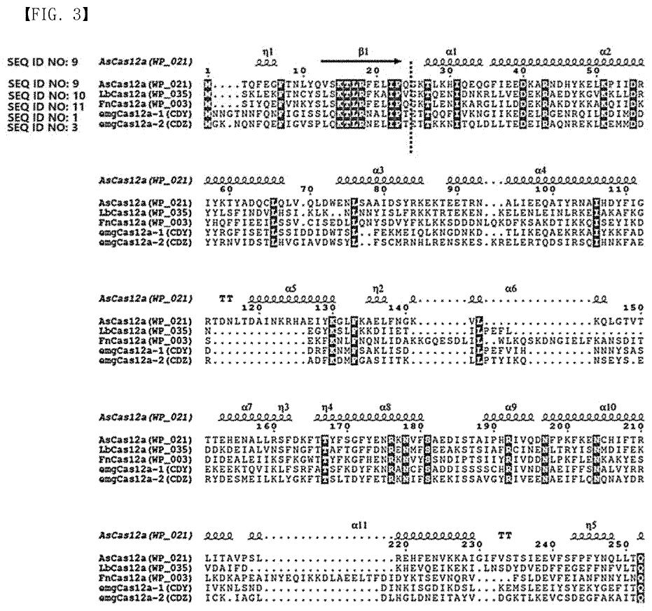

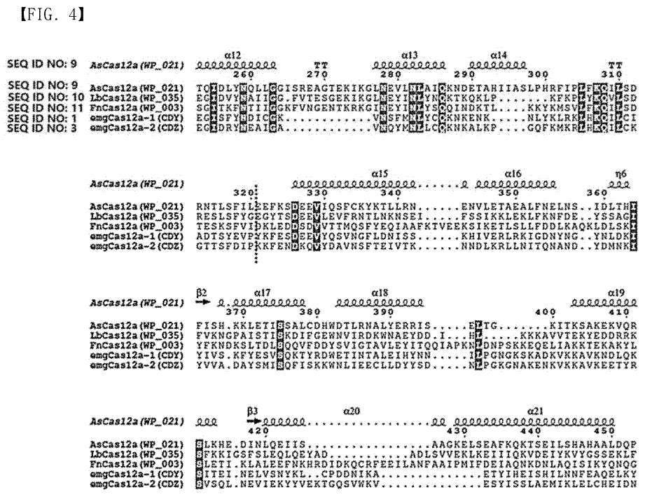

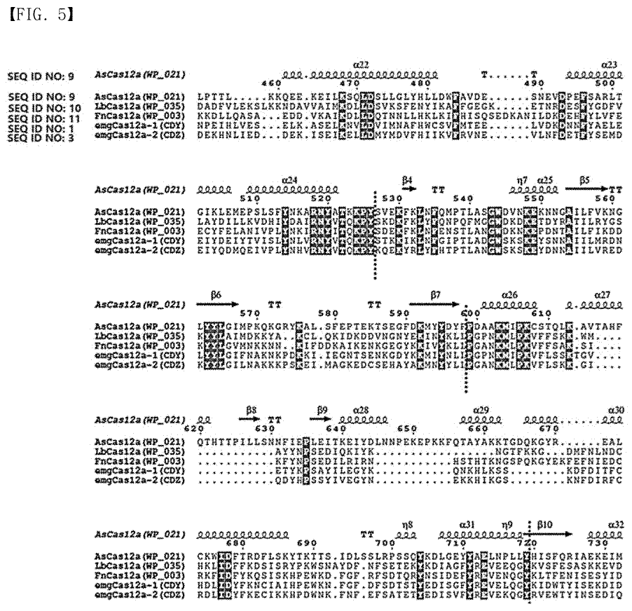

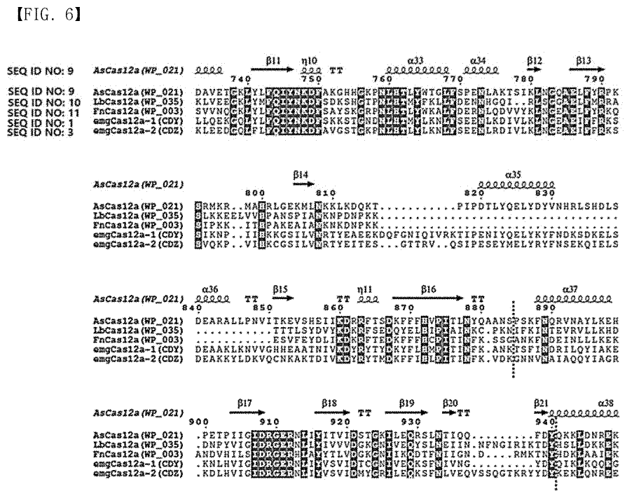

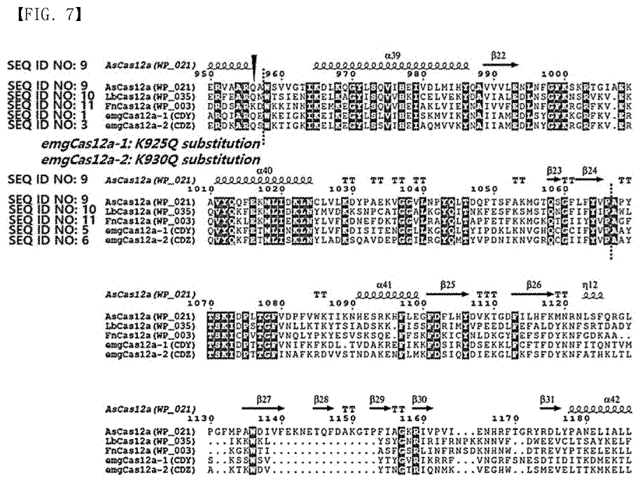

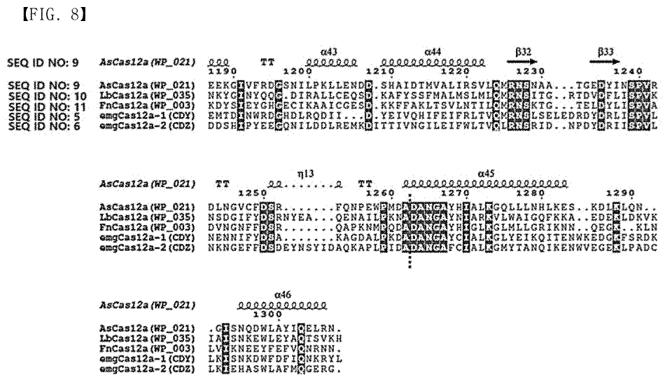

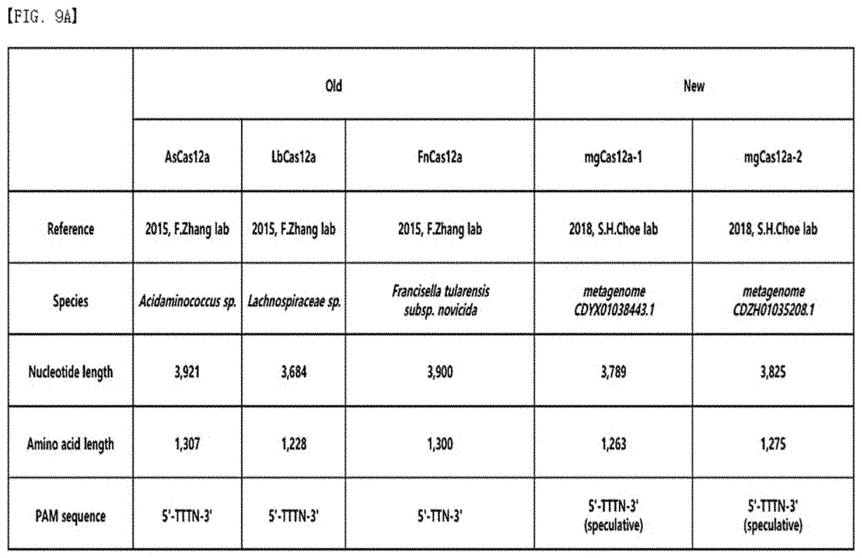

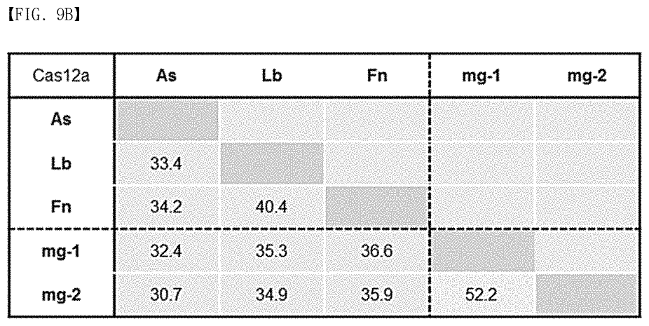

Cas12a candidates were aligned based on the structures of AsCas12a and LbCas12a using the ESPript program. For the WT mgCas12a-1 and WT mgCas12a-2, substitution of part of the amino acids was made to increase their endonuclease activity. The WT mgCas12a-1, in which the 925^{th} amino acid Lys(K) was substituted with Glu(Q), was named mgCas12a-1. In addition, the WT mgCas12a-2, in which the 930^{th} amino acid Lys(K) was substituted with Glu(Q), was named mgCas12a-2. The resulting variants were subjected to codon optimization in consideration of codon usages of humans, Arabidopsis, and E. coli, and then a request for gene synthesis thereof was made to Bionics. Here, the nucleotide sequences of the human codon-optimized mgCas12a-1 and mgCas12a-2 are shown in SEQ ID NO: 7 and SEQ ID NO: 8, respectively. In addition, the amino acid sequences of the existing Cas12a's (AsCas12a (SEQ ID NO: 9), LbCas12a (SEQ ID NO: 10), and FnCas12a (SEQ ID NO: 11)) and the Cas12a candidates (mgCas12a-1 and mgCas12a-2), which were aligned using the ESPript program, are illustrated in FIGS. 3 to 8; and the results obtained by comparing and summarizing their sequence information are illustrated in FIGS. 9A and 9B.

Then, each of the WT mgCas12a-1, WT mgCas12a-2, mgCas12a-1, and mgCas12a-2 genes, which had been cloned into pUC57 vector, was again inserted into pET28a-KanR-6×His-BPNLS vector, and then cloning was performed. The cloned vector was transformed into the E. coli strains DH5a and Rosetta, respectively. A 5′-handle sequence of crRNA was extracted from the metagenome CRISPR repeat sequence. The extracted RNA was synthesized into a DNA oligo. Transcription of the DNA oligomer was performed using the MEGAshortscript T7 RNA transcriptase kit, and a concentration of the transcribed 5′-handle was checked by FLUOstar Omega.

### Example 3. Protein Expression and Purification

5 ml of the E. coli Rosetta (DE3), which was cultured overnight, was inoculated into 500 ml of liquid TB medium supplemented with 100 mg/ml of kanamycin antibiotic. The medium was cultured in an incubator at 37° C. until the OD600 reached 0.6. For protein expression, treatment with 0.4 uM of isopropyl β-D-1-thiogalactopyranoside (IPTG) was performed, and then further culture was performed at 22° C. for 16 to 18 hours. After centrifugation, the obtained cells were mixed with 10 ml of lysis buffer (20 mM HEPES pH 7.5, 100 mM KCl, 20 mM imidazole, 10% glycerol, and EDTA-free protease inhibitor cocktail), and then subjected to ultrasonication for cell disruption. The disruption was centrifuged three times at 6,000 rpm for 20 minutes each, and then filtered through a 0.22 micron filter.

Thereafter, washing and elution were performed using a nickel column (HisTrap FF, 5 ml) and 300 mM imidazole buffer, and the proteins were purified by affinity chromatography. The protein sizes were checked by SDS-PAGE electrophoresis, and dialysis was performed overnight against dialysis buffer (20 mM HEPES pH 7.5, 100 mM KCl, 1 mM DTT, 10% glycerol). Then, the proteins were selectively subjected to filtration and concentration (Amicon Ultra Centrifugal Filter 100,000 MWCO) depending on their size. For the proteins, Bradford quantitative method was used to measure their concentration. Then, the proteins were stored at −80° C. and used.

### Example 4. Identification of pH Range Suitable for mgCas12a Through Cleavage Analysis

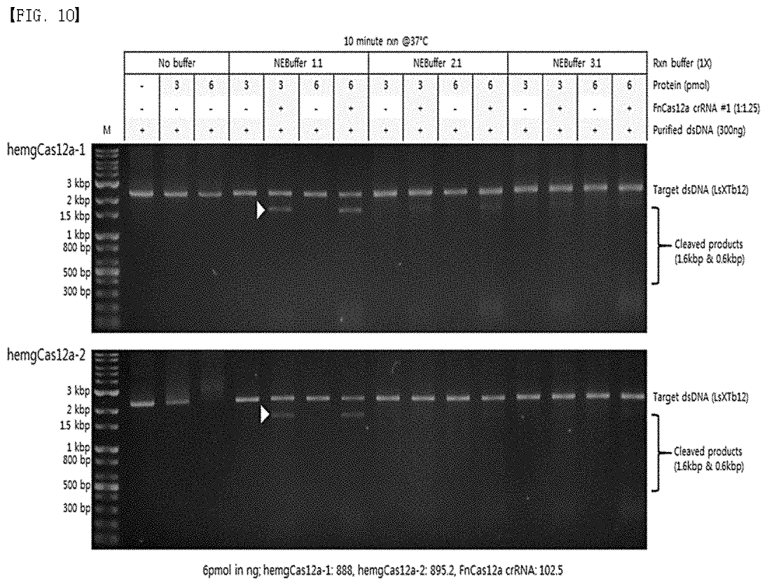

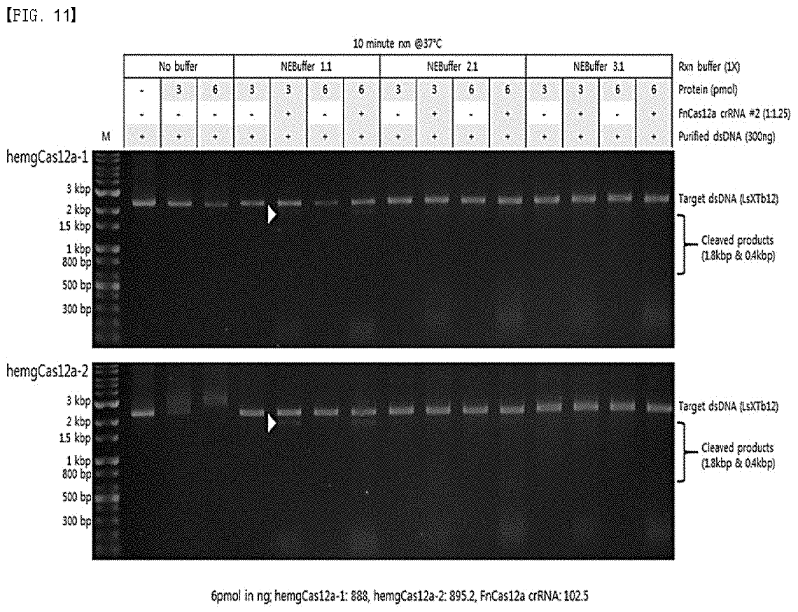

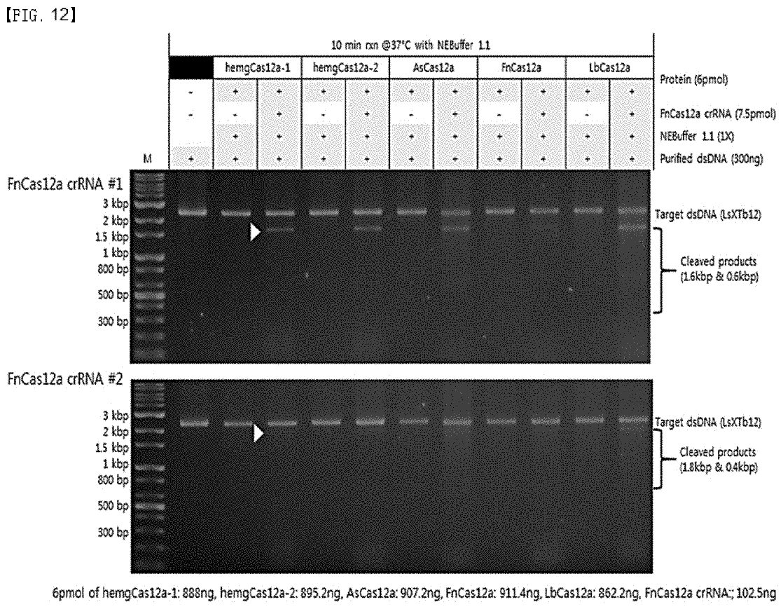

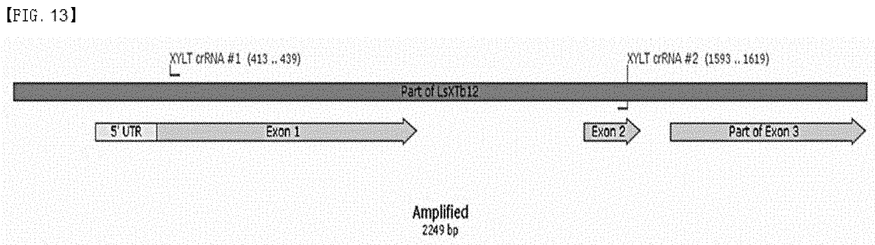

Xylosyltransferase of lettuce (Lactuca sativa) was amplified by PCR to predict a protospacer adjacent motif (PAM), and a guide RNA (gRNA) therefor was designed. For ribonucleoprotein (RNP) complexes for mgCas12a-1 and mgCas12a-2, each mgCas12a protein was mixed with the gRNA at a molecular ratio of 1:1.25 at room temperature for 20 minutes, to produce each RNP complex. The purified xylosyltransferase PCR product was subjected to treatment with the RNPs at various concentrations. Then, concentration adjustment was conducted with NEBuffer 1.1 (1× Buffer Components, 10 mM Bis-Tris-Propane-HCl, 10 mM MgCl₂ and 100 μg/ml BSA), NEBuffer 2.1 (1× Buffer Components, 50 mM NaCl, 10 mM Tris-HCl, 10 mM MgCl₂ and 100 μg/ml BSA), and NEBuffer 3.1 (1× Buffer Components, 100 mM NaCl, 50 mM Tris-HCl, 10 mM MgCl₂ and 100 μg/ml BSA), and an in vitro cleavage analysis was performed at 37° C. Here, the NEBuffer 1.1, the NEBuffer 2.1, and the NEBuffer 3.1 had pH 7.0, pH 7.9, and pH 7.9 values, respectively, at 25° C. After each reaction was completed, the reaction was stopped by incubation at 65° C. for 10 minutes, and the completed reaction was checked by 1.5% agarose gel electrophoresis. The results are illustrated in FIGS. 10 to 12. In FIGS. 10 to 12, the mgCas12a-1 and the mgCas12a-2 are designated by hemgCas12a-1 and hemgCas12a-2, respectively. In addition, the target nucleic acid sequence, which is in the xylosyltransferase, and the positions where the crRNAs bind were indicated in a diagram, and this diagram is illustrated in FIG. 13.

As illustrated in FIGS. 10 to 12, in a case where the mgCas12a-1 and crRNA complex was treated with the NEBuffer 1.1, the target dsDNA was cleaved. In addition, in a case where the mgCas12a-2 and crRNA complex was treated with the NEBuffer 1.1, the target dsDNA was cleaved. From these results, it was found that the mgCas12a-1 and mgCas12a-2 were active at pH 7.0.

### Example 5. Analysis of Gene Editing Efficiency of mgCas12a in Animal Cells

### Example 5.1. Production of RNP Including mgCas12a-1 or mgCas12a-2 for Gene Editing of CCR5 and DNMT1

HEK 293T cells were cultured in a 5% CO₂ incubator at 37° C. in DMEM medium supplemented with 10% fetal bovine serum (FBS) and penicillin-streptomycin (P/S). Each 100 pmole of the mgCas12a-1 protein and the mgCas12a-2 protein, and 200 pmole of each of CCR5-targeting crRNA and DNMT1-targeting crRNA were incubated at room temperature for 20 minutes, to prepare each RNP. Here, the crRNA sequences for CCR5 and DNMT1 were synthesized by Integrated DNA Technologies (IDT), and are shown in Table 1 below.

TABLE 1

| Genes | crRNA sequence (5′-3′) |
|---|---|
| CCR5 | CACCGAAUUUCUACUGUUGUAGAUGGAGUGAAGGGAGAGUUUGU |
| | CAAUUUUUUG (SEQ ID NO: 12) |
| DNMT1 | GGUCAAUUUCUACUGUUGUAGAUGCUCAGCAGGCACCUGCCUCU |
| | UUU (SEQ ID NO: 13) |

The cultured HEK293T cells at 2×10⁵ were mixed with 20 μl of nucleofection reagent, and then mixed with 10 μl of RNP complex. Subsequently, 4D-Nucleofector device (Lonza) was used for transfection. 48 and 72 hours after transfection, genomic DNA was extracted from the cells using PureLink™ Genomic DNA Mini Kit (Invitrogen).

### Example 5.2. Sequencing Analysis for Target Site

The genomic DNA extracted in Example 5.1 was amplified using adapter primers for CCR5 or DNMT1 shown in Table 2 below.

TABLE 2

| Genes | Adapter primer sequence (5′-3′) |
|---|---|
| CCR5 | TCGTCGGCAGCGTCAGATGTGTATAAGAGACAGGGTATTTCTG |
| | TTCAGATCAC (SEQ ID NO: 15) |
| | GTCTCGTGGGCTCGGAGATGTGTATAAGAGACAGGCCCATCAA |
| | TTATAGAAAGCC (SEQ ID NO: 16) |
| DNMT1 | TCGTCGGCAGCGTCAGATGTGTATAAGAGACAGCTGCACACAG |
| | CAGGCCTTTG (SEQ ID NO: 17) |
| | GTCTCGTGGGCTCGGAGATGTGTATAAGAGACAGCCCAATAAG |
| | TGGCAGAGTGC (SEQ ID NO: 18) |

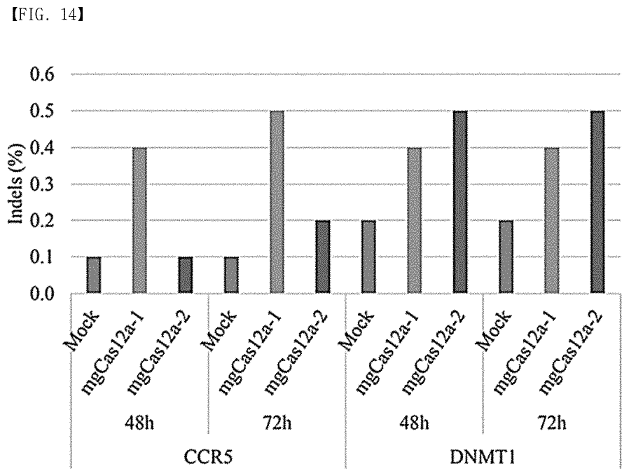

Subsequently, purification and sequencing library preparation were performed according to the protocol of Illumina, and then a deep-sequencing analysis was performed on the target site using MiniSeq equipment. The gene editing efficiency achieved by the mgCas12a-1 and mgCas12a-2 proteins is illustrated in FIG. 14, and the sequencing analysis results for the target site are shown in Table 3 below. As illustrated in FIG. 14, the mgCas12a-1 and mgCas12a-2 proteins exhibited higher gene editing efficiency than that of the mock protein.

TABLE 3

| | | | | | With both | More than | | | | Indel |
|---|---|---|---|---|---|---|---|---|---|---|
| | | | | Total | indicator | minimum | | | Indel | frequency |
| Samples | Genes | Time | Name | Sequences | sequences | frequency | Insertions | Deletions | frequency | (%) |
| 1 | CCR5 | 48 h | Mock | 137952 | 137475 | 137196 | 0 | 187 | 187 (0.1%) | 0.1 |
| 2 | | | mgCas12a-1 | 119684 | 119250 | 118952 | 36 | 418 | 454 (0.4%) | 0.4 |
| 3 | | | mgCas12a-2 | 112387 | 112077 | 111826 | 8 | 150 | 158 (0.1%) | 0.1 |
| 4 | | 72 h | Mock | 139323 | 138942 | 138647 | 8 | 179 | 187 (0.1%) | 0.1 |
| 5 | | | mgCas12a-1 | 156795 | 156159 | 155857 | 39 | 738 | 777 (0.5%) | 0.5 |
| 6 | | | mgCas12a-2 | 158717 | 158392 | 158048 | 5 | 237 | 242 (0.2%) | 0.2 |
| 7 | DNMT1 | 48 h | Mock | 141182 | 136856 | 136469 | 19 | 316 | 335 (0.2%) | 0.2 |
| 8 | | | mgCas12a-1 | 122368 | 120871 | 120476 | 70 | 424 | 494 (0.4%) | 0.4 |
| 9 | | | mgCas12a-2 | 121928 | 120592 | 120218 | 46 | 509 | 555 (0.5%) | 0.5 |
| 10 | | 72 h | Mock | 98480 | 96480 | 96170 | 0 | 192 | 192 (0.2%) | 0.2 |
| 11 | | | mgCas12a-1 | 126317 | 123792 | 123370 | 2 | 511 | 513 (0.4%) | 0.4 |
| 12 | | | mgCas12a-2 | 47398 | 47999 | 46738 | 12 | 199 | 211 (0.5%) | 0.5 |

### Example 6. Analysis of Gene Editing Efficiency of mgCas12a in Plant Cells

### Example 6.1. Plant Protoplast Isolation

Tobacco seeds were sterilized by treatment with 50% Clorox for 1 minute. The sterilized seeds were placed on a medium for seed germination and cultured for a week. Then, the seeds were transferred to a magenta box used for culture, and grown for 3 weeks. The light culture condition used was 16 hours of light and 8 hours of darkness, and the seeds were grown at a temperature of 25° C. to 28° C. For the plant, leaves grown for 4 to 6 weeks were used. The leaf was placed on a glass plate, and the leaf apex and petiole were cut therefrom so that only an inner part of the leaf was used. Here, the leaf was cut into pieces of 0.5 mm or smaller. The cut leaf pieces were placed in 10 mL of Enzyme solution and incubated on an orbital shaker (50 rpm) at room temperature for 3 to 4 hours in the dark.

After incubation, 10 mL of W5 solution was added and carefully mixed. A cell strainer (70 μm) was used to filter the protoplasts present in the Enzyme solution. The filtered protoplasts were centrifuged at 100×g for 6 minutes. The supernatant was discarded, and the protoplast pellet was carefully suspended by addition of MMG solution. Then, the suspension was placed on ice for 10 to 30 minutes. For a part of the suspension, the number of protoplasts was counted using a Hem cytometer, which is a counter plate, and a microscope. Subsequently, MMG solution was further added for dilution so that the protoplast concentration reached 2×10⁶ cells/mL. The composition for each of the enzyme solution, MMG solution, and PEG solution is shown in Table 4 below.

TABLE 4

| Enzyme solation | 20 | mL |
|---|---|---|
| 1.0% Cellulase R10 | 200 | mg |
| 0.5% Macerozyme R10 | 100 | mg |
| 0.4M Mannitol | 10 | mL (0.8M mannitol stock solution) |
| 20 mM MRS, pH 5.7 | 4 | mL (100 mM MES stock solution, pH 5.7) |
| 20 mM KCl | 200 | μL (2M KCl stock solution) |

Combination of the above-mentioned reagents is performed, incubation is performed for 10 minutes
at 60° C., and then combination with the following reagents is performed.

| 10 mM CaCl₂•2H₂O | 200 | μL (1M CaCl₂•2H₂O stock solution) |
|---|---|---|
| 0.1% BSA | 200 | μL (10% BSA stock solution) |
| MMG solution | 10 | mL |
| 0.4M Mannitol | 5 | mL (0.8M mannitol stock solution) |
| 4 mM MBS, pH 5.7 | 400 | μL (0.1M MES stock solution, pH 5.7) |
| 15 mM MgCl₂ | 150 | μL (1M MgCl₂ stock solution) |
| Nuclease-free water | 4.45 | mL |
| PEG solution | 5 | mL |
| 0.2M Mannitol | 1.25 | mL (0.8M mannitol stock solution) |
| 40% W/V PEG-4000 | 2 | g (polyethylene glycol 4000) |
| 100 mM CaCl₂•2H₂O | 500 | μL (1M CaCl₂•2H₂O stock solution) |
| Nuclease-free water | 1.5 | mL |
| W5 solution | 50 | mL |
| 154 mM NaCl | 3.85 | mL (2M NaCl stock solution) |
| 125 mM CaCl₂•2H₂O | 6.25 | mL (1M CaCl₂•2H₂O stock solution) |
| 5 mM KCl | 125 | μL (2M KCl stock solution) |
| 2 mM MES, pH 5.7 | 500 | μL (0.1M MES stock solution) |
| Nuclease-free water | 39.275 | mL |

### Example 6.2. Sequencing Analysis for Target Site and Identification of Editing Efficiency Therefor

crRNA, mgCas12a protein, and NEB buffer 1.1 were added to a 2 mL e-tube to a final volume of 20 μL, and then reaction was allowed to proceed at room temperature for 10 minutes. 200 μL (5×10⁵ cells) of the protoplast obtained in Example 6.1, and the reacted crRNA and mgCas12 protein (volume 20 μL) were added to an e-tube (2 mL), mixed well, and then cultured for 10 minutes in a clean bench. Subsequently, 220 μL of PEG solution, which was the same volume as the incubated volume, was added thereto and carefully mixed. The mixture was cultured at room temperature for 15 minutes. Then, 840 μL of W5 solution was added thereto and mixed well. Ater centrifugation at 100×g for 2 minutes, the supernatant was discarded. Then, culture was performed in W5 solution for two days. Then, the cells were harvested and DNA was extracted therefrom.

Using the extracted DNA, the target portion was subjected to PCR, and then the target gene editing efficiency was identified by next-generation sequencing (NGS). The results are shown in Table 5 below. As shown in Table 5, the gene editing efficiency achieved by the mgCas12a-1 protein was 1.8-fold higher than that of FnCpf1.

TABLE 5

| | | | | With both | More than | | | | |
|---|---|---|---|---|---|---|---|---|---|
| Target | | | Total | indicator | minimum | | | Indel | |
| gene | crRNA | Nuclease | Sequences | sequences | frequency | Insertions | Deletions | frequency | |
| FucT14-1 | 2 | none | 161551 | 161421 | 160896 | 4 | 180 | 184 | (0.1%) |
| | | mgCas12a-1 | 124361 | 124255 | 123844 | 3 | 168 | 171 | (0.1%) |
| | | mgCas12a-2 | 99154 | 99053 | 98734 | 0 | 131 | 131 | (0.1%) |
| | | FnCpf1 | 50060 | 50022 | 49808 | 0 | 63 | 63 | (0.1%) |
| | 4 | none | 161551 | 161411 | 160899 | 4 | 178 | 182 | (0.1%) |
| | | mgCas12a-1 | 106782 | 106706 | 106330 | 0 | 1877 | 1877 | (1.8%) |
| | | mgCas12a-2 | 126665 | 126544 | 126057 | 79 | 885 | 964 | (0.8%) |
| | | FnCpf1 | 64554 | 64501 | 64272 | 15 | 470 | 485 | (0.8%) |
| FucT14-2 | 2 | none | 49459 | 49422 | 49192 | 2 | 49 | 51 | (0.1%) |
| | | mgCas12a-1 | 81191 | 81101 | 80738 | 0 | 90 | 90 | (0.1%) |
| | | mgCas12a-2 | 83694 | 83614 | 83286 | 0 | 99 | 99 | (0.1%) |
| | | FnCpf1 | 108803 | 108682 | 108260 | 0 | 112 | 112 | (0.1%) |
| | 4 | none | 49459 | 49427 | 49199 | 2 | 49 | 51 | (0.1%) |
| | | mgCas12a-1 | 54918 | 54854 | 54532 | 6 | 689 | 695 | (1.3%) |
| | | mgCas12a-2 | 127825 | 127691 | 127213 | 2 | 143 | 145 | (0.1%) |
| | | FnCpf1 | 64265 | 64168 | 63882 | 0 | 162 | 162 | (0.3%) |

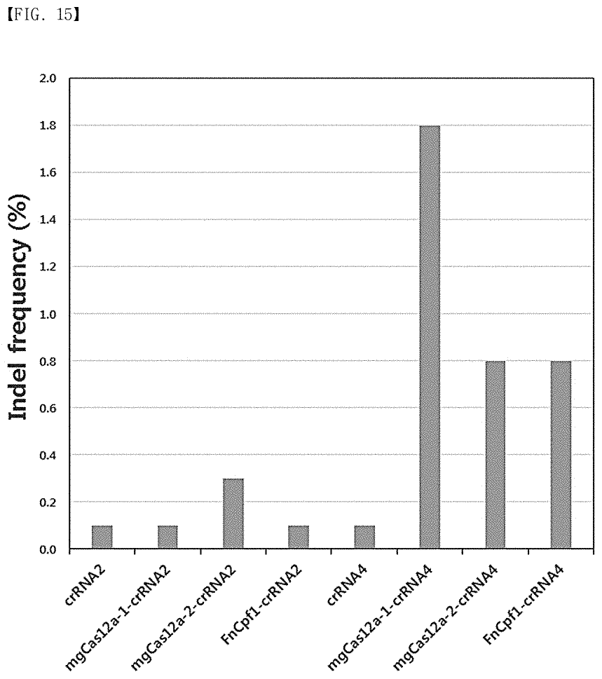

In addition, the gene editing efficiency achieved by using two crRNAs for the tobacco FucT14 genes was identified for each protein. The results are illustrated in FIG. 15. As illustrated in FIG. 15, the gene editing efficiency achieved by the mgCas12a-1 protein was 2-fold higher than that of FnCpf1. Here, the crRNAs and primer sequences for the target genes NbFucT14_1 and NbFucT14_2 are shown in Tables 6 and 7 below.

| | crRNA | crRNA sequence |
|---|---|---|
| Target Gene | (primer name) | (PAM site) |
| NbFucT14_1 | NbFTa14_1/2-2 | TTTGGATAATTTGTACTCTTGTCG |
| NbFucT14_2 | | ATGT (SEQ ID NO: 19) |
| | NbFTa14_1/2-4 | TTTAGTCCACAAACAGCTAAGCCC |
| | | ACAT (SEQ ID NO: 20) |

| | | | Size |
|---|---|---|---|
| Target gene | Primer name | Sequence | (bp) |
| NbFucT14_1 | NGS NbFTa14_1_F | TGAGCTGAAGATGGATTATG | 216 |
| | | (SEQ ID NO: 21) | |
| | NGS NbFTa14_1_R | TCATGCTTAAGATAAAAGAG | |
| | | (SEQ ID NO: 22) | |
| NbFucT14_2 | NGS NbFTa14_2_F | TCATGAGCTTAAGATGGATC | 217 |
| | | (SEQ ID NO: 23) | |
| | NGS NbFTa14_2_R | GTTTAAGCTAAAAGAACTAC | |
| | | (SEQ ID NO: 24) | |

### Example 7. Comparison of Gene Editing Efficiency Between FnCas12a and mgCas12a

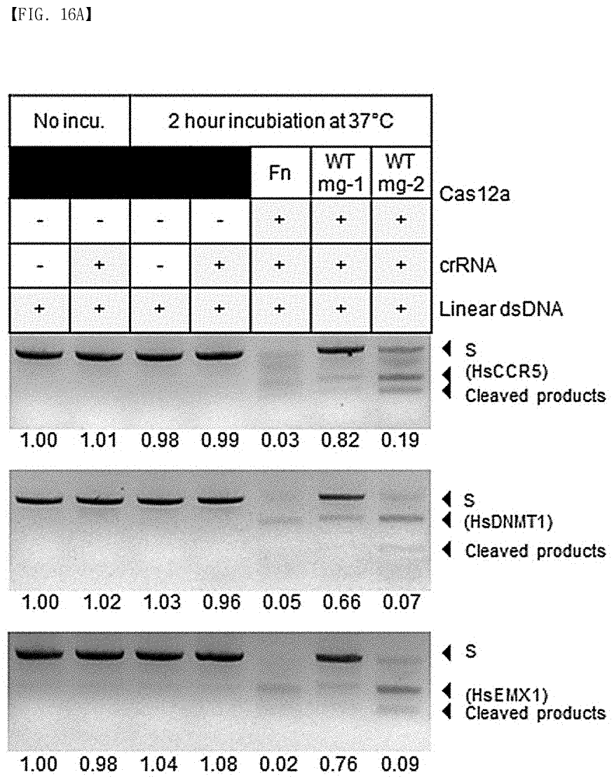

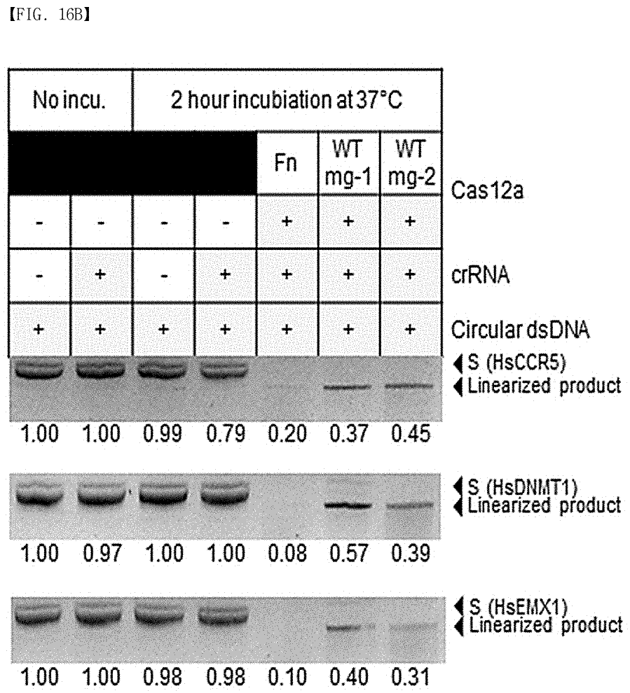

To form each ribonucleoprotein (RNP) complex consisting of FnCas12a, WT mgCas12a-1 or WT mgCas12a-2 protein, and crRNA, 6 pmol of FnCas12a, WT mgCas12a-1, or WT mgCas12a-2 protein, and 7.5 pmol of crRNA were mixed with NEB1.1 buffer and 1× distilled water at room temperature for 30 minutes. To identify dsDNA cleavage activity using the crRNA-dependent Cas12a (FnCas12a, WT mgCas12a-1, or WT mgCas12a-2), 0.3 pmol of target dsDNA (linear or circular) was added thereto, and then reaction was allowed to proceed at 37° C. for 2 hours. Here, HsCCR5, HsDNMT1, and HsEMX1 were used as DNA. In addition, the linear DNAs (SEQ ID NO: 27 to SEQ ID NO: 29) used in the experiment were PCR purified products, and the circular DNAs (SEQ ID NO: 30 to SEQ ID NO: 32) were purified plasmids. SDS and EDTA (gel loading dye, NEB) were added thereto, and then the mixture was stored at −20° C. for 10 minutes to stop the reaction. Each DNA was loaded on a 1% agarose gel, and then subjected to electrophoresis to check the DNA cleavage activity caused by the FnCas12a, WT mgCas12a-1, or WT mgCas12a-2. The results are illustrated in FIGS. 16A (linear DNA) and 16B (circular DNA). In FIGS. 16A and 16B, S denotes a substrate, and each number indicated at the bottom of the gel denotes how dark the substrate DNA band is.

### Example 8. Identification of Non-Specific DNase Activity of mgCas12a

To identify random DNase functions of the Cas12a (AsCas12a, FnCas12a, or LbCas12a) and the mgCas12a (WT mgCas12a-1, d_mgCas12a-1, WrmgCas12a-2, or d_mgCas12a-2), an experiment was performed in the same manner as in Example 7. Here, the d-mgCas12a-1 and the d_mgCas12a-2 refer to proteins obtained from the WT mgCas12a-1 and the WT mgCas12a-2, respectively, by substitution of Asp (at position 877 for the WT mgCas12a-1 or at position 873 for the WT mgCas12a-2) with Ala.

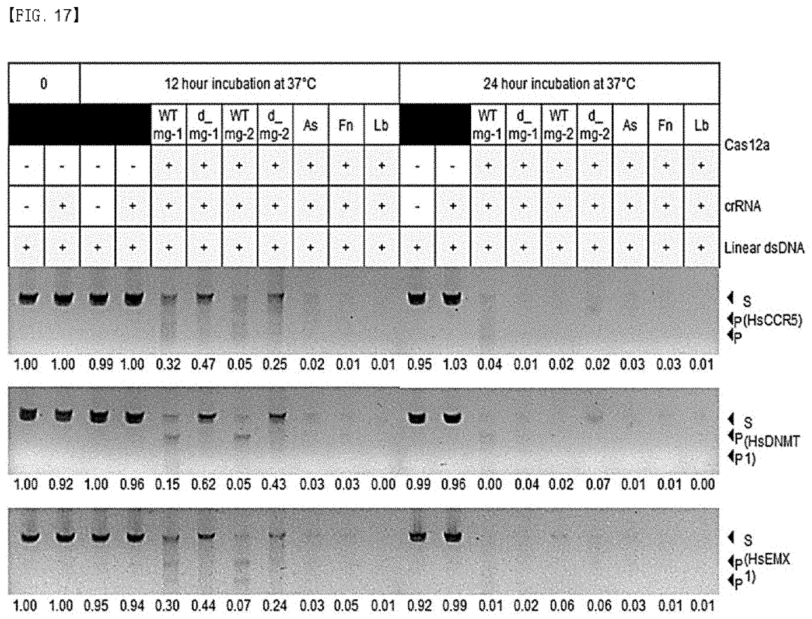

Specifically, to form each ribonucleoprotein (RNP) complex consisting of each of the 7 types of Cas12a and crRNA, 6 pmol of each Cas12a protein and 7.5 pmol of crRNA were allowed to react at room temperature for 30 minutes in the presence of NEB1.1 buffer and 1× distilled water. Subsequently, 0.3 pmol of target dsDNA was added thereto, and then reaction was allowed to proceed at 37° C. for 12 hours or 24 hours. Here, HsCCR5, HsDNMT1, and HsEMX1 were used as DNA. SDS and EDTA (gel loading dye, NEB) were added thereto, and then the mixture was stored at −20° C. for 10 minutes to stop the reaction. Each DNA was loaded on a 1% agarose gel, and then subjected to electrophoresis to check the DNA cleavage activity caused by the 7 types of Cas12a. The results are illustrated in FIG. 17. In FIG. 17, S denotes a substrate, and each number indicated at the bottom of the gel denotes how dark the substrate DNA band is.

As illustrated in FIG. 17, each ribonucleoprotein complex consisting of the WT mgCas12a-1, d_mgCas12a-1, WTmgCas12a-2, or d_mgCas12a-2, which is novel Cas12a, and crRNA exhibited a weaker non-specific DNase function than the ribonucleoprotein complex consisting of the AsCas12a, FnCas12a, or LbCas12a, which is existing Cas12a, and crRNA. In addition, overall, it could be presumed that reaction of the Cas12a RNP with DNA results in a non-specific DNase function.

### Example 9. Identification of Non-Specific DNase Function of Cas12a Under crRNA-Free Condition

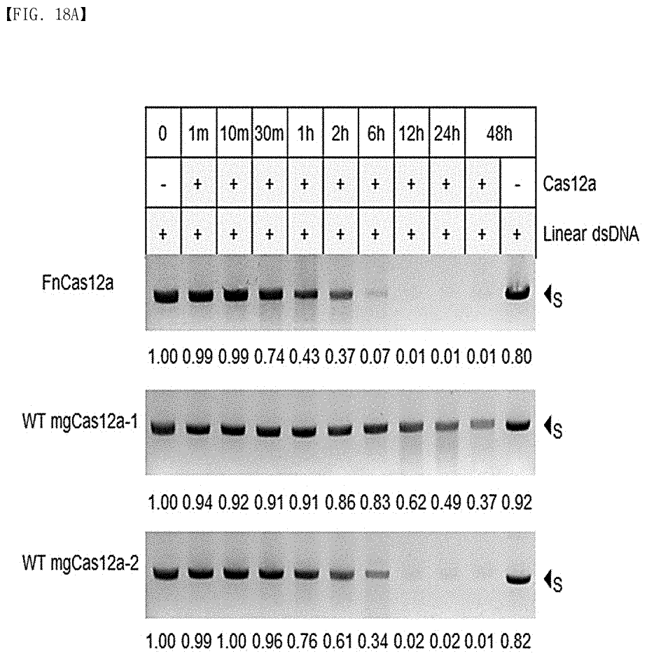

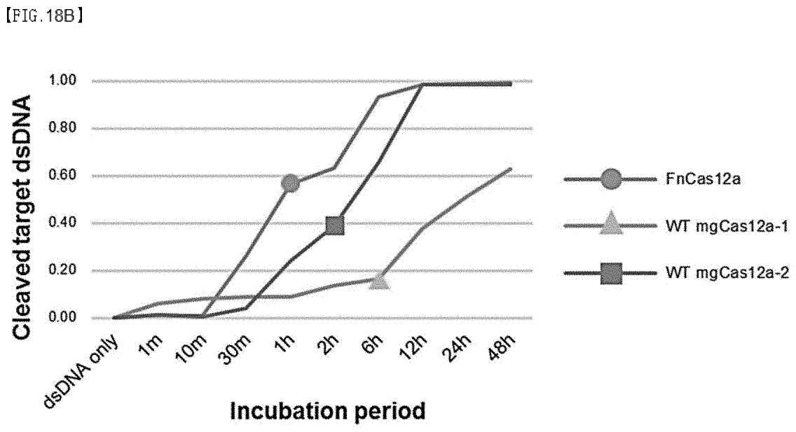

To identify whether Cas12a has a random DNase function even without crRNA, for the FnCas12a, WT mgCas12a-1, or WT mgCas12a-2 protein, an experiment was performed in the same manner as in Example 7 with varying times, except that a crRNA-free condition was used. The results are illustrated in FIGS. 18A and 18B. As illustrated in FIGS. 18A and 18B, the FnCas12a, WT mgCas12a-1, or WT mgCas12a-2 protein had a random DNase function even without crRNA, in which the random DNase function of the FnCas12a protein appeared first.

### Example 10. Identification of DNA Cleavage Function of mgCas12a Using Handle of Existing Cas12a

To identify whether the new Cas12a (d_mgCas12a or WT mgCas12a) can perform DNA cleavage using a handle located at the 5′ end of the existing Cas12a (AsCas12a, FnCas12a, or LbCas12a) sequence, an experiment was performed in the same manner as in Example 7 with varying reaction times, except that the handle of each of the AsCas12a, FnCas12a, or LbCas12a was used. The results are illustrated in FIG. 19.

As illustrated in FIG. 19, in a case where DNA cleavage was performed with the d_mgCas12a or WT mgCas12a protein using the handle of the AsCas12a, FnCas12a or LbCas12a, all d_mgCas12a or WT mgCas12a proteins using the three types of handles had a DNA cleavage function, although the DNA cleavage efficiency was slightly different depending on the respective handles. From these results, it was found that for DNA cleavage, the mgCas12a can use the handle of the AsCas12a, FnCas12a, or LbCas12a.

### Example 11. Identification of Activity of FnCas12a or mgCas12a in Divalent Ions

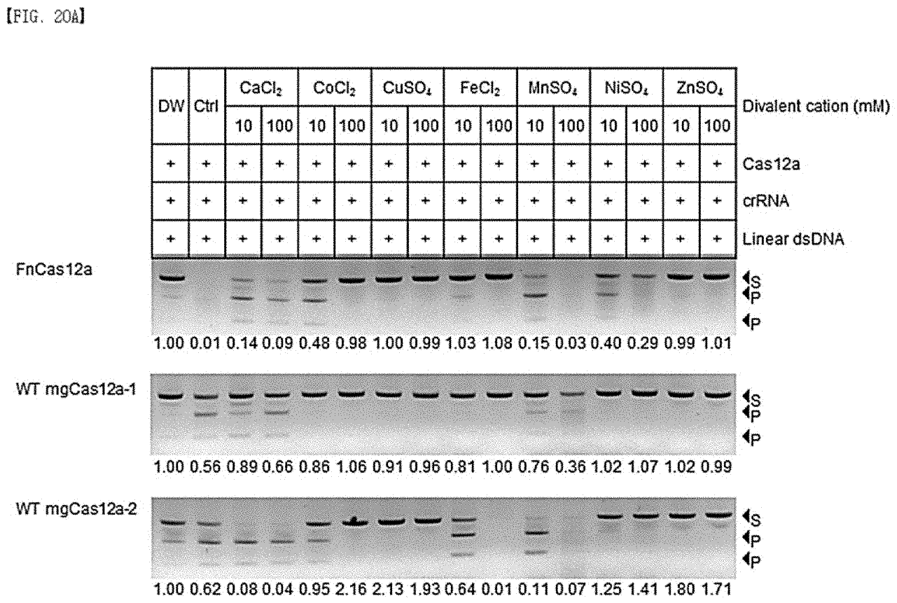

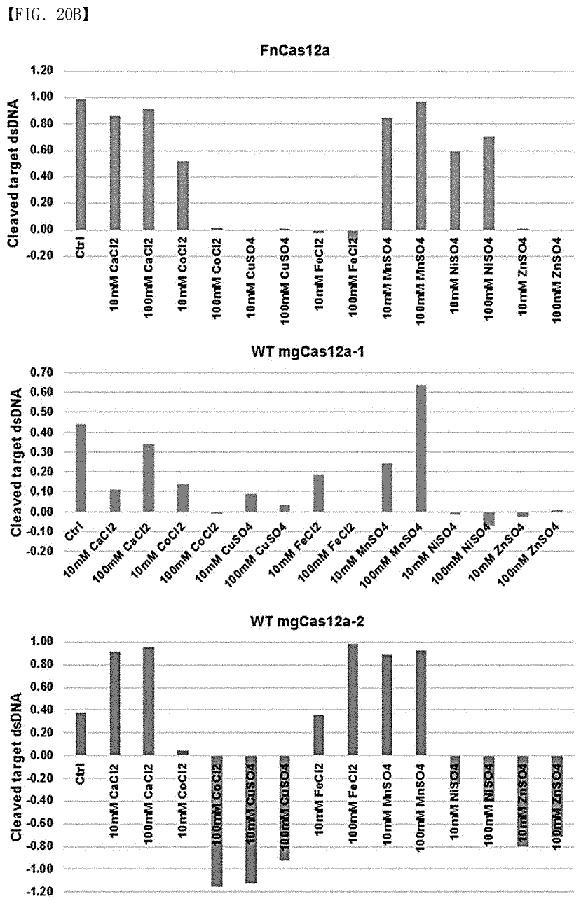

In addition, to identify DNA cleavage activity of the FnCas12a, mgCas12a-1, or mgCas12a-2 protein in divalent ions (CaCl₂, CoCl₂, CuSO₄, FeCl₂, MnSO₄, NiSO₄, or ZnSO₄), an experiment was performed in the same manner as in Example 4, except that a predetermined amount of divalent ions was used in place of the NEBuffer 1.1. The results are illustrated in FIGS. 20A and 20B. As illustrated in FIGS. 20A and 20B, the FnCas12a, mgCas12a-1, or mgCas12a-2 protein exhibited similar DNA cleavage activity in the same divalent ions.

1. A Cas12a protein comprising the amino acid sequence of SEQ ID NO: 1.

2. The Cas12a protein of claim 1, wherein the Cas12a protein comprising the amino acid sequence of SEQ ID NO: 1 is encoded by the nucleotide sequence of SEQ ID NO: 2.

3. The Cas12a protein of claim 1, wherein the protein has endonuclease activity.

4. The Cas12a protein of claim 1, wherein the Cas12a protein comprising the amino acid sequence of SEQ ID NO: 1 has optimal activity at pH 7.0 to pH 7.9.

5. A Cas12a protein comprising the amino acid sequence of SEQ ID NO: 1, of which lysine (Lys) at position 925 is substituted with another amino acid.

6. The Cas12a protein of claim 5, wherein the other amino acid is any one selected from the group consisting of arginine (Arg), histidine (His), aspartic acid (Asp), glutamic acid (Glu), serine (Ser), threonine (Thr), asparagine (Asn), glutamine (Gln), tyrosine (Tyr), alanine (Ala), isoleucine (Ile), leucine (Leu), valine (Val), phenylalanine (Phe), methionine (Met), tryptophan (Trp), glycine (Gly), proline (Pro), and cysteine (Cys).

7. A Cas12a protein comprising the amino acid sequence of SEQ ID NO: 3.

8. The Cas12a protein of claim 7, wherein the Cas12a protein comprising the amino acid sequence of SEQ ID NO: 3 is encoded by the nucleotide sequence of SEQ ID NO: 4.

9. The Cas12a protein of claim 7, wherein the protein has endonuclease activity.

10. The Cas12a protein of claim 7, wherein the Cas12a protein comprising the amino acid sequence of SEQ ID NO: 3 has optimal activity at pH 7.0 to pH 7.9.

11. A Cas12a protein comprising the amino acid sequence of SEQ ID NO: 3, of which lysine (Lys) at position 930 is substituted with another amino acid.

12. The Cas12a protein of claim 11, wherein the other amino acid is any one selected from the group consisting of arginine (Arg), histidine (His), aspartic acid (Asp), glutamic acid (Glu), serine (Ser), threonine (Thr), asparagine (Asn), glutamine (Gln), tyrosine (Tyr), alanine (Ala), isoleucine (Ile), leucine (Leu), valine (Val), phenylalanine (Phe), methionine (Met), tryptophan (Trp), glycine (Gly), proline (Pro), and cysteine (Cys).

13. A Cas12a protein comprising the amino acid sequence of SEQ ID NO: 1, of which aspartic acid (Asp) at position 877 is substituted with another amino acid.

14. The Cas12a protein of claim 13, wherein the other amino acid is any one selected from the group consisting of arginine (Arg), histidine (His), glutamic acid (Glu), serine (Ser), threonine (Thr), asparagine (Asn), glutamine (Gln), tyrosine (Tyr), alanine (Ala), lysine (Lys), isoleucine (Ile), leucine (Leu), valine (Val), phenylalanine (Phe), methionine (Met), tryptophan (Trp), glycine (Gly), proline (Pro), and cysteine (Cys).

15. The Cas12a protein of claim 13, wherein the protein has decreased endonuclease activity.

16. A Cas12a protein comprising the amino acid sequence of SEQ ID NO: 3, of which aspartic acid (Asp) at position 873 is substituted with another amino acid.

17. The Cas12a protein of claim 16, wherein the other amino acid is any one selected from the group consisting of arginine (Arg), histidine (His), glutamic acid (Glu), serine (Ser), threonine (Thr), asparagine (Asn), glutamine (Gln), tyrosine (Tyr), alanine (Ala), lysine (Lys), isoleucine (Ile), leucine (Leu), valine (Val), phenylalanine (Phe), methionine (Met), tryptophan (Trp), glycine (Gly), proline (Pro), and cysteine (Cys).

18. The Cas12a protein of claim 16, wherein the protein has decreased endonuclease activity.

19. A pharmaceutical composition for treating cancer, comprising as active ingredients:
mgCas12a; and
crRNA that targets a nucleic acid sequence specifically present in cancer cells.

20. The pharmaceutical composition of claim 19, wherein the mgCas12a has any one amino acid sequence selected from the group consisting of SEQ ID NO: 1, SEQ ID NO: 3, SEQ ID NO: 5, and SEQ ID NO: 6.


SEQUENCE LISTING (32 sequences)

SEQ ID NO: 1 - protein, 1263 aa, Artificial Sequence
  mgCas12a-1
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQAEKRKAIYKKFADDDRFKNMFSAKLISDILPEFVIHNNNYSASEKEEKTQVIKLFSRFATSFKDYFKNRANCFSADDISSSSCHRIVNDNAEIFFSNALVYRRIVKNLSNDDINKISGDIKDSLKEMSLEEIYSYEKYGEFITQEGISFYNDICGKVNSFMNLYCQKNKENKNLYKLRKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCPDDNIKAETYIHEISHILNNFEAQELKYNPEIHLVESELKASELKNVLDVIMNAFHWCSVFMTEELVDKDNNFYAELEEIYDEIYTVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHLKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKTIPENIYQELYKYFNDKSDKELSDEAAKLKNVVGHHEAATNIVKDYRYTYDKYFLHMPITINFKANKTSFINDRILQYIAKEKNLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKKLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 2 - DNA, 3792 bp, Artificial Sequence
  mgCas12a-1
atgaataacggaacaaataactttcagaactttatcggaatttcttctttgcagaagactcttaggaatgctctcattccaacagaaacaacacagcaatttattgttaaaaatggaataattaaagaagatgaactcagaggagaaaatcgtcagatacttaaagatatcatggatgattattacagaggtttcatttcagaaactttatcgtcaattgatgatattgactggacctctttatttgagaaaatggaaattcagttaaaaaatggagataataaagacactcttataaaagaacaggctgaaaaacgtaaggcaatctataaaaaatttgcagatgatgatagatttaaaaatatgttcagtgcaaaattaatctcagatattcttcctgaatttgtcattcataacaataattattctgcatcagaaaaggaagaaaaaacacaggtaattaaattattttccagatttgcaacatcattcaaggactattttaaaaacagggctaattgtttttctgctgatgatatatcttcttcttcttgtcatagaatagttaatgataatgcagaaatattttttagtaatgcattggtgtataggagaattgtaaaaaatctttcaaatgatgatataaataaaatatccggagatattaaggattcattaaaggaaatgtctctggaggaaatttattcttatgaaaaatatggggaatttattacacaggaaggtatatctttttataatgatatatgcggtaaagtaaattcatttatgaatttatattgccagaaaaataaagaaaacaaaaatctctataagctgcgaaagcttcataaacagatactgtgcatagcagatacttcttatgaggtgccgtataaatttgaatcagatgaagaggtttatcaatcagtgaatggatttttggacaatattagttcaaaacatatcgttgaaagattgcgtaagattggagacaactataacggctacaatcttgataagatttatattgttagtaaattctatgaatcagtttcacaaaagacatatagagattgggaaacaataaatactgcattagaaattcattacaacaatatattacccggaaatggtaaatctaaagctgacaaggtaaaaaaagcggtaaagaatgatctgcaaaaaagcattactgaaatcaatgagcttgttagcaattataaattatgtccggatgataatattaaagcagagacatatatacatgaaatatcacatattttgaataattttgaagcacaggagcttaagtataatcctgaaattcatctggtggaaagtgaattgaaagcatctgaattaaaaaatgttctcgatgtaataatgaatgcttttcattggtgttcggttttcatgacagaggagctggtagataaagataataatttttatgcggagttagaagagatatatgacgaaatatatacggtaatttcattgtataatcttgtgcgtaattatgtaacgcagaagccatatagtacaaaaaaaattaaattgaattttggtattcctacactagcggatggatggagtaaaagtaaagaatatagtaataatgcaattattctcatgcgtgataatttgtactatttaggaatatttaatgcaaaaaataagcctgacaaaaagataattgaaggtaatacatcagaaaataaaggggattataagaagatgatttataatcttctgccaggaccaaataaaatgatccccaaggtattcctctcttcaaaaaccggagtggaaacatataagccgtctgcctatatattggagggctataaacaaaacaagcatcttaaatcctctaaggattttgatataacgttttgtcacgatttgattgattattttaagaactgtatagcaatacatcctgaatggaagaattttggctttgatttttctgacacctccacatatgaagatatcagcggattttacagagaagtcgaattgcaaggttataaaattgactggacatatatcagcgaaaaggatattgatttgttgcaggaaaaaggacagttatatttatttcaaatatataacaaagatttttccaagaaaagtaccggaaatgataatcttcatactatgtatttgaagaatttgtttagcgaagagaatttaaaggatattgtactgaaattaaacggtgaggcggaaatcttctttagaaaatcaagcataaagaatccaataattcataaaaaaggctctattcttgttaatagaacatatgaagcagaggaaaaagatcaatttggaaatatccagatagtcagaaaaaccataccggaaaatatatatcaggagctttataaatatttcaatgataaaagtgataaagaactttcggatgaagcagctaagcttaagaatgtagtaggtcatcatgaggctgctacaaacatagtaaaagattatagatatacatatgataaatattttcttcatatgcctattacaatcaattttaaagccaataagacaagctttattaatgacagaatattacaatatattgctaaagaaaagaatttgcatgtaataggcattgatcgtggtgaaagaaacctgatatatgtttcagtaattgatacttgtggaaatattgttgaacaaaaatcgtttaacattgttaatggatatgattatcagattaagctcaagcagcaggagggggcgcgacaaatcgcacgaaaagaatggaaagaaatcggcaaaataaaagaaattaaagaaggctatttatctcttgtaattcatgaaatttcaaagatggttattaaatataatgccataattgcaatggaggatttaagctacggatttaaaaaaggtcgtttcaaggttgagcgacaggtttaccagaagtttgagacaatgcttatcaacaaactcaactatctggtatttaaagatatatccataactgaaaacggtggtcttctaaagggatatcagcttacatatattccagataaactgaaaaatgtgggtcatcaatgtggttgtatattttacgtacctgctgcctatacatcaaaaatagatcctacaaccggatttgtaaatatattcaaatttaaagatttaacagttgatgcaaagagagaatttataaaaaaatttgacagtatcagatatgattcagaaaaaaaactgttttgttttacatttgattataataactttattacgcaaaatactgttatgtcaaagtcaagctggagtgtatatacgtacggagttaggataaaaagaagatttgtcaatggcaggttctcaaatgaatcggatacaattgatataacaaaagatatggaaaaaaccctcgaaatgacagatataaattggagagatggtcatgatctgaggcaggatattattgattatgaaatcgtacaacacatatttgagatttttagattgactgtacaaatgagaaacagtttaagtgaattagaagacagggattatgaccgtttgatttctccggtgctcaatgaaaataatatattttatgattcagctaaagcaggagatgcgttacctaaagacgcagatgctaatggtgcatattgtatagctctaaaaggcttgtatgaaatcaaacaaattacagagaattggaaagaagacggtaagttttcaagagataaacttaaaatttccaataaggactggtttgactttattcaaaataaaaggtatttataa

SEQ ID NO: 3 - protein, 1275 aa, Artificial Sequence
  mgCas12a-2
MGKNQNFQEFIGVSPLQKTLRNELIPTETTKKNITQLDLLTEDEIRAQNREKLKEMMDDYYRNVIDSTLHVGIAVDWSYLFSCMRNHLRENSKESKRELERTQDSIRSQIHNKFAERADFKDMFGASIITKLLPTYIKQNSEYSERYDESMEILKLYGKFTTSLTDYFETRKNIFSKEKISSAVGYRIVEENAEIFLQNQNAYDRICKIAGLDLHGLDNEITAYVDGKTLKEVCSDEGFAKAITQEGIDRYNEAIGAVNQYMNLLCQKNKALKPGQFKMKRLHKQILCKGTTSFDIPKKFENDKQVYDAVNSFTEIVTKNNDLKRLLNITQNANDYDMNKIYVVADAYSMISQFISKKWNLIEECLLDYYSDNLPGKGNAKENKVKKAVKEETYRSVSQLNEVIEKYYVEKTGQSVWKVESYISSLAEMIKLELCHEIDNDEKHNLIEDDEKISEIKELLDMYMDVFHIIKVFRVNEVLNFDETFYSEMDEIYQDMQEIVPLYNHVRNYVTQKPYKQEKYRLYFHTPTLANGWSKSKEYDNNAIILVREDKYYLGILNAKKKPSKEIMAGKEDCSEHAYAKMNYYLLPGANKMLPKVFLSKKGIQDYHPSSYIVEGYNEKKHIKGSKNFDIRFCRDLIDYFKECIKKHPDWNKFNFEFSATETYEDISVFYREVEKQGYRVEWTYINSEDIQKLEEDGQLFLFQIYNKDFAVGSTGKPNLHTLYLKNLFSEENLRDIVLKLNGEAEIFFRKSSVQKPVIHKCGSILVNRTYEITESGTTRVQSIPESEYMELYRYFNSEKQIELSDEAKKYLDKVQCNKAKTDIVKDYRYTMDKFFIHLPITINFKVDKGNNVNAIAQQYIAGRKDLHVIGIDRGERNLIYVSVIDMYGRILEQKSFNLVEQVSSQGTKRYYDYKEKLQNREEERDKARKSWKTIGKIKELKEGYLSSVIHEIAQMVVKYNAIIAMEDLNYGFKRGRFKVERQVYQKFETMLISKLNYLADKSQAVDEPGGILRGYQMTYVPDNIKNVGRQCGIIFYVPAAYTSKIDPTTGFINAFKRDVVSTNDAKENFLMKFDSIQYDIEKGLFKFSFDYKNFATHKLTLAKTKWDVYTNGTRIQNMKVEGHWLSMEVELTTKMKELLDDSHIPYEEGQNILDDLREMKDITTIVNGILEIFWLTVQLRNSRIDNPDYDRIISPVLNKNGEFFDSDEYNSYIDAQKAPLPIDADANGAFCIALKGMYTANQIKENWVEGEKLPADCLKIEHASWLAFMQGERG

SEQ ID NO: 4 - DNA, 3828 bp, Artificial Sequence
  mgCas12a-2
atgggtaaaaatcaaaattttcaggaatttattggggtatcaccacttcaaaagactttaagaaacgaattaatcccaacagaaacaacaaaaaagaatattactcagcttgatcttttgactgaggatgaaatccgcgcgcaaaatcgagagaagctgaaagagatgatggatgactactaccggaatgtgattgatagcactttgcatgtgggtatagctgttgattggagctatttattttcgtgtatgcgaaatcatctaagggagaattccaaagagtcaaagcgggaattggaacgaacacaggattctattcgttcacaaatccataataagtttgctgaacgagcggattttaaggatatgtttggagcatcgataataacaaaattacttccgacatatataaaacagaattcagaatattccgagcggtatgacgagagcatggaaattttgaaactgtatggaaaattcacaacatcgttgaccgattactttgagacaagaaagaatatcttttctaaagagaaaatatcttctgccgttggatatcgaatcgtagaggaaaatgctgagatcttcttgcagaatcagaatgcttacgacagaatctgtaagatagcgggactggatttacatggattggataatgaaataacagcatatgttgatggaaaaacattaaaagaagtatgttcggatgaaggatttgcaaaggctattacacaagaagggattgatcgctacaacgaggcaatcggtgcagtaaatcaatatatgaatctgttatgccagaagaataaggcattaaaaccgggacaatttaagatgaagcggctacataaacagattctttgcaaaggaacaacctctttcgatattccaaagaagtttgaaaatgataaacaggtgtatgacgcagttaattcttttacagagatagtaacgaagaataatgatttgaagcgactgttaaatattacacagaatgcaaatgattatgacatgaataaaatctatgtagtagccgatgcatatagtatgatttcacagtttatcagtaaaaaatggaatctgattgaagaatgcttgctggattattatagcgataatttgccgggaaaaggaaatgcgaaagaaaacaaagttaaaaaggcggtaaaggaagaaacgtatcgcagtgtttcacagttgaatgaagttattgagaaatattatgtggaaaagaccggacagtcagtatggaaagtggaaagttatatttctagtctggcagaaatgattaagctggaattgtgccacgagatagataacgatgagaagcataatctgattgaagatgatgagaagatatccgagattaaggaactgttggatatgtacatggatgtatttcatattataaaagtgttccgggtgaatgaagtattgaatttcgatgaaaccttttattcggagatggatgagatctatcaggatatgcaggaaatcgttccattatacaatcatgttcgaaactatgttacacagaaaccatataagcaggagaaatatcgtttatatttccacactccaacattggcaaatggctggtccaagagtaaggaatatgacaacaacgcaattatattggtgcgagaagataaatattatttaggtattctgaatgcgaaaaagaaaccatcgaaagaaattatggcgggcaaagaggattgttcagaacatgcatatgcaaagatgaattattatttgttgccgggcgcgaacaagatgcttccaaaagtatttttatctaagaaaggaatacaggactatcacccatcatcatatattgttgaaggatataatgaaaagaaacatattaaaggttccaagaattttgatatccggttttgtagggatttgattgactacttcaaggaatgcattaaaaaacatccggattggaataagtttaactttgaattttctgcgacagaaacatatgaggatatcagtgtcttttatcgcgaagttgaaaagcaaggatatcgcgtagagtggacgtatatcaatagtgaagatattcagaaactggaagaagatggacagttgtttttatttcagatatataacaaagattttgctgtgggaagtacaggtaaaccaaatcttcatacattgtatctgaaaaatctgttcagcgaagaaaatttgcgggacattgtattaaaactaaatggggaagcagaaatattcttccgtaaatcaagtgttcaaaaaccggtgattcataagtgcggcagtattttagtcaatcgtacctatgagattaccgagagtggaacaacacgggtacaatcaattccggaaagtgaatacatggaattatatcgctactttaatagtgaaaagcagatagaattatcagatgaggcaaaaaaatatttggacaaggtgcaatgtaataaggcaaagacagatattgtgaaagactaccgatacaccatggacaagttttttattcatcttccgattacgattaattttaaggttgataagggtaacaatgttaatgccattgcacagcaatatattgcagggcggaaagatttacatgtgataggaattgatcgaggagaacggaatctgatttacgtttctgtaattgacatgtatggtagaattttagagcagaaatcctttaaccttgtggaacaggtatcgtcgcagggaacgaagcgatattacgattacaaagaaaaattacagaaccgggaagaggaacgggataaagcaagaaagagttggaagacaatcggcaagattaaggaattaaaagaggggtatctgtcgtcagtaattcatgagattgcacagatggtcgtaaagtataacgcaatcattgcaatggaagatttgaattatggatttaagcggggaagattcaaagtagagcgccaggtatatcagaaatttgaaacgatgttgatcagtaagttgaattatctggcagataaatctcaggctgtggatgaaccgggaggtatattacggggatatcagatgacttatgtgccggataatattaagaatgttggaagacaatgtggaataatcttttatgtgccggcagcatatacctccaagattgatccgacaaccggatttatcaatgcatttaagcgggatgtggtatcaacaaatgatgcaaaagagaatttcctgatgaagtttgattctattcagtacgatatagaaaaaggcttatttaagttttcatttgattacaaaaattttgccacacataaacttacacttgcgaagacaaaatgggacgtatatacaaatggaactcgaatacaaaacatgaaagttgaaggacattggctttcaatggaagttgaacttacaacgaaaatgaaagagttgctggatgactcgcatattccatatgaagaaggacagaatatattggatgatttgcgggagatgaaagatataacaaccattgtgaatggtatattggaaatcttctggttgacagtccagcttcggaatagcaggatagataatccggattacgatagaattatctcaccggtattgaataaaaatggagaattttttgattctgatgaatataattcatatattgatgcgcaaaaggcaccgttaccgatagatgccgatgcaaatggcgcattttgcattgcattaaaaggaatgtatactgccaatcagatcaaagaaaactgggttgaaggggagaaacttccggcggattgcttgaagatcgaacatgcgagttggttagcatttatgcaaggagaaaggggatag

SEQ ID NO: 5 - protein, 1263 aa, Artificial Sequence
  engineered mgCas12a-1(K925Q)
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQAEKRKAIYKKFADDDRFKNMFSAKLISDILPEFVIHNNNYSASEKEEKTQVIKLFSRFATSFKDYFKNRANCFSADDISSSSCHRIVNDNAEIFFSNALVYRRIVKNLSNDDINKISGDIKDSLKEMSLEEIYSYEKYGEFITQEGISFYNDICGKVNSFMNLYCQKNKENKNLYKLRKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCPDDNIKAETYIHEISHILNNFEAQELKYNPEIHLVESELKASELKNVLDVIMNAFHWCSVFMTEELVDKDNNFYAELEEIYDEIYTVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHLKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKTIPENIYQELYKYFNDKSDKELSDEAAKLKNVVGHHEAATNIVKDYRYTYDKYFLHMPITINFKANKTSFINDRILQYIAKEKNLHVIGIDRGERNLIYVSVIDTCGNIVEQKSFNIVNGYDYQIKLKQQEGARQIARQEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKKLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 6 - protein, 1275 aa, Artificial Sequence
  engineered mgCas12a-2(K930Q)
MGKNQNFQEFIGVSPLQKTLRNELIPTETTKKNITQLDLLTEDEIRAQNREKLKEMMDDYYRNVIDSTLHVGIAVDWSYLFSCMRNHLRENSKESKRELERTQDSIRSQIHNKFAERADFKDMFGASIITKLLPTYIKQNSEYSERYDESMEILKLYGKFTTSLTDYFETRKNIFSKEKISSAVGYRIVEENAEIFLQNQNAYDRICKIAGLDLHGLDNEITAYVDGKTLKEVCSDEGFAKAITQEGIDRYNEAIGAVNQYMNLLCQKNKALKPGQFKMKRLHKQILCKGTTSFDIPKKFENDKQVYDAVNSFTEIVTKNNDLKRLLNITQNANDYDMNKIYVVADAYSMISQFISKKWNLIEECLLDYYSDNLPGKGNAKENKVKKAVKEETYRSVSQLNEVIEKYYVEKTGQSVWKVESYISSLAEMIKLELCHEIDNDEKHNLIEDDEKISEIKELLDMYMDVFHIIKVFRVNEVLNFDETFYSEMDEIYQDMQEIVPLYNHVRNYVTQKPYKQEKYRLYFHTPTLANGWSKSKEYDNNAIILVREDKYYLGILNAKKKPSKEIMAGKEDCSEHAYAKMNYYLLPGANKMLPKVFLSKKGIQDYHPSSYIVEGYNEKKHIKGSKNFDIRFCRDLIDYFKECIKKHPDWNKFNFEFSATETYEDISVFYREVEKQGYRVEWTYINSEDIQKLEEDGQLFLFQIYNKDFAVGSTGKPNLHTLYLKNLFSEENLRDIVLKLNGEAEIFFRKSSVQKPVIHKCGSILVNRTYEITESGTTRVQSIPESEYMELYRYFNSEKQIELSDEAKKYLDKVQCNKAKTDIVKDYRYTMDKFFIHLPITINFKVDKGNNVNAIAQQYIAGRKDLHVIGIDRGERNLIYVSVIDMYGRILEQKSFNLVEQVSSQGTKRYYDYKEKLQNREEERDKARQSWKTIGKIKELKEGYLSSVIHEIAQMVVKYNAIIAMEDLNYGFKRGRFKVERQVYQKFETMLISKLNYLADKSQAVDEPGGILRGYQMTYVPDNIKNVGRQCGIIFYVPAAYTSKIDPTTGFINAFKRDVVSTNDAKENFLMKFDSIQYDIEKGLFKFSFDYKNFATHKLTLAKTKWDVYTNGTRIQNMKVEGHWLSMEVELTTKMKELLDDSHIPYEEGQNILDDLREMKDITTIVNGILEIFWLTVQLRNSRIDNPDYDRIISPVLNKNGEFFDSDEYNSYIDAQKAPLPIDADANGAFCIALKGMYTANQIKENWVEGEKLPADCLKIEHASWLAFMQGERG

SEQ ID NO: 7 - DNA, 3789 bp, Artificial Sequence
  human codon optimized engineered mgCas12a-1
atgaacaatggcaccaacaatttccagaactttatcggaattagcagtctgcaaaagactctccggaatgcccttatacccaccgagacaacccagcagttcatcgtgaaaaacgggattatcaaggaagacgagctgcgcggcgaaaatcggcaaattttgaaagatataatggacgattattaccgcggttttatctctgagactctgagctccattgacgatatcgactggacctcactcttcgaaaagatggagattcagcttaaaaacggcgataataaggacacactgataaaagaacaggctgagaagcggaaagccatctataagaaatttgcagatgacgatcgcttcaagaacatgtttagcgccaaattgattagtgacatcctgccggaattcgttattcacaataacaattactctgctagcgagaaggaagagaaaacccaagtcataaagctcttttcccggttcgccacttcatttaaagattatttcaagaaccgcgcaaattgctttagcgccgacgatatcagttctagctcctgtcatcggattgtgaacgacaatgctgaaatcttcttttcaaacgcccttgtataccgccggattgtgaaaaatctgagcaacgatgacataaataagatcagtggagatattaaagactctttgaaggagatgagcctggaagagatctattcctacgaaaaatatggggagttcattacccaggaaggcatatcattttacaacgatatctgcggtaaggttaatagcttcatgaacctctattgtcagaaaaataaggagaacaaaaatctttacaagctgcgcaaattgcacaagcaaattctgtgcatcgcagacacaagttatgaagtcccttacaaatttgagtctgatgaagaggtgtatcagagcgtaaacggcttcctcgacaatatttcctcaaagcatatagtggaacggcttcgcaaaatcggagataactacaatgggtataacctggacaagatttacatcgttagcaaattttatgagagtgtctctcagaagacctaccgggattgggaaactattaataccgccttggagatacactataacaatatcctgcccggcaacggtaaaagcaaggctgacaaagtgaagaaagccgtaaagaatgatctccaaaaatccattacagaaatcaacgagcttgtgtcaaattacaagctgtgtccggacgataacattaaagcagaaacctatatacatgagatcagccacattttgaataacttcgaagcccaggagctgaagtacaatccagaaatccatctcgttgagagtgaacttaaagcttctgagctgaagaacgtcttggacgtgattatgaatgcctttcactggtgcagcgtattcatgactgaagagctggtggataaagacaacaatttttatgcagaactcgaggaaatatacgatgagatctataccgttatttccctttacaacctggtccgcaattatgtgacacagaagccctactcaaccaaaaagatcaaattgaacttcggcattccgactctggccgacggatggagcaagagtaaagaatattctaataacgctataatcctcatgcgggataatctttactatctggggatttttaacgccaagaataaacctgacaagaaaatcattgagggcaacaccagcgaaaataagggtgattacaaaaagatgatatataacttgctgcccggcccgaataaaatgatcccaaaggtattcctctcctcaaaaacaggagtggagacctacaagcccagcgcatatattcttgaagggtacaaacaaaacaagcatctgaaaagttctaaggactttgatatcactttctgtcacgacttgattgattattttaaaaattgcatagccatccatccggagtggaagaacttcggctttgacttcagcgatacctccacatacgaagacatttcaggtttttatcgcgaggttgaactgcagggctacaaaatcgattggacctatattagcgagaaggacatagatctccttcaggaaaaaggacaactgtacttgttccagatctataataaggactttagtaaaaagtctactgggaacgataatctgcacaccatgtacctcaaaaaccttttcagcgaggaaaatctgaaggacattgtcttgaaactgaacggcgaggctgaaatctttttccggaagtcctcaattaaaaatcctataatccataagaaaggtagcattctcgtgaaccgcacatatgaggccgaagagaaggatcagtttggcaatatccaaattgtacggaaaaccatacccgaaaacatctaccaggagctttataagtacttcaatgacaaaagtgataaggaactgtctgacgaggcagccaaattgaagaacgtggttggacaccatgaagctgccactaatattgtcaaagattatcgctacacctatgacaagtactttctgcacatgccgatcacaattaacttcaaagcaaataagaccagctttataaacgatcggattctccagtatattgccaaagagaagaatcttcatgtgatcgggattgaccgcggcgaacggaacctgatatacgtatccgtgatcgatacttgtggtaatattgttgagcaaaaatcattcaacatcgtcaatggctatgactaccagattaagttgaaacagcaagaaggagctcgccagatagcccggcaggagtggaaggaaatcgggaaaattaaggagatcaaagaaggctatctgagcctcgtgattcacgagataagtaagatggtaatcaaatacaacgcaattatcgccatggaagatctttcttatggttttaagaaaggccgcttcaaggtggagcggcaagtttaccagaaatttgaaaccatgctgattaataagttgaactatctggtcttcaaagacataagcatcacagagaatggagggctccttaagggctaccagctgacctatattccagataaattgaagaacgtgggtcatcaatgcggctgtatcttttacgtacccgctgcctatacttccaaaattgacccgaccacaggattcgtgaatatatttaagttcaaagatctgaccgttgacgcaaagcgcgaatttatcaaaaagttcgattcaattcggtacgacagcgagaaaaagctcttttgcttcacttttgattataacaatttcatcacccagaacacagtcatgagtaaatctagctggtccgtgtacacctatggggtacgcattaagcggcgctttgtgaatggccggttctcaaacgaaagcgacactatagatatcaccaaagacatggagaagacacttgaaatgaccgatattaattggcgcgacggtcacgatctgcggcaggacatcattgattacgagatagttcaacatatctttgaaattttccgcttgactgtccagatgcggaacagtctgtctgagctcgaagaccgcgattatgaccggcttatcagccctgtgctgaatgagaacaatattttttacgattccgccaaagctggcgacgccttgcccaaggatgcagacgccaacggagcttattgtatagccctgaaagggctctacgaaatcaagcagattaccgagaattggaaagaagatggcaagttctcacgcgacaaacttaagatcagcaacaaagattggtttgacttcattcaaaataagcggtatctg

SEQ ID NO: 8 - DNA, 3825 bp, Artificial Sequence
  human codon optimized engineered mgCas12a-2
atgggcaaaaaccaaaatttccaagaatttatcggagtgagccccctgcagaagaccctccggaacgagcttattccgactgagaccacaaagaaaaatataacccagctggacttgctgactgaagatgagatccgcgcccagaaccgggaaaagctcaaagagatgatggacgattattaccgcaatgttattgacagtacccttcacgtcgggatcgctgtggattggtcttatctgttcagctgcatgcggaaccatttgcgcgaaaattccaaggagtcaaaacgggaactggagcgcacacaggacagcattcggagtcagatacacaacaagtttgccgaacgcgcagatttcaaagacatgtttggcgcctctatcattaccaagctccttcctacttacatcaaacaaaatagcgagtattccgaacggtacgatgagtcaatggaaattctgaagttgtatggtaaattcaccacaagcctgaccgactactttgagactcgcaagaacatattcagtaaagaaaagatctctagcgctgtaggctatcggattgtggaggaaaatgccgagatctttctccagaaccagaatgcatacgatcgcatttgtaaaatagccggacttgacctgcatgggttggataacgaaatcaccgcttatgttgacggcaagacactgaaagaggtctgctccgatgaaggtttcgccaaggcaattacccaagagggcatcgaccggtacaatgaagccattggagctgtgaaccagtatatgaatctcctttgtcagaaaaacaaggccctgaaacccgggcaatttaagatgaaacgcttgcacaagcagatactgtgcaaaggcactacctcattcgatatcccgaagaaatttgagaatgacaagcaggtatacgatgcagtgaacagcttcacagaaattgttaccaaaaataacgacctcaagcggcttctgaatatcactcaaaacgccaatgattatgacatgaacaaaatttacgtcgtggctgatgcctatagtatgatatctcagtttatcagcaagaaatggaatttgattgaggaatgtctgctcgactactattccgataaccttccaggtaagggcaatgcaaaagagaacaaggtaaaaaaggccgtgaaagaagagacctaccgctcagttagccagctgaatgaagtcatcgagaagtattacgtggaaaaaacaggacaaagtgtatggaaggtggagtcttatattagctccttggctgaaatgataaaactggagctctgccatgaaatcgacaacgatgagaagcacaatcttattgaagacgatgagaaaatctcagaaattaaggagctgttggacatgtacatggatgttttccatataatcaaagtctttcgggtgaacgaagtactgaatttcgacgagaccttttatagcgaaatggatgagatttaccaggacatgcaggaaatcgtgcccctctataaccacgttcgcaattacgtcactcaaaagccgtataaacaggagaagtaccggctttatttccatacccctacactggccaacgggtggagtaaatctaaggaatacgataataacgcaattatattggtgcgcgaggacaaatattacctgggcatcctcaatgccaagaaaaagcccagcaaagaaattatggctggtaaggaggattgttccgaacacgcctatgcaaaaatgaactactatcttctgccgggcgccaataagatgttgccaaaagtatttctgtcaaagaaaggaatccaggactaccatcccagcagttatattgtggaggggtacaacgaaaagaaacacataaagggctctaaaaatttcgatatccggttttgccgcgacctcattgattatttcaaggagtgtatcaaaaagcatccggactggaacaaatttaatttcgaatttagcgctaccgagacttacgaagatatttccgttttctatcgggaggtcgaaaagcaaggttaccgcgtggagtggacctatataaactcagaagacatccagaaacttgaggaagatggccagctgtttttgttccaaatttacaataaggactttgccgtaggaagcacagggaaacctaacctgcacaccctctatcttaagaatctgttcagtgaggaaaacttgcgggatatcgtgctgaaactcaatggcgaggcagaaatttttttccgcaagtctagcgttcagaaacccgtcatacataagtgcggttccatccttgtgaaccggacttacgagattaccgaatcaggcacaacccgcgtacagagcatcccggagagtgaatatatggagctgtaccggtattttaattctgaaaaacaaattgagttgagcgacgaagccaagaaatacctggataaggtgcagtgtaacaaagctaagactgacatagttaaagattatcgctacaccatggacaagttctttatccacctcccaattacaatcaatttcaaagtcgataagggaaacaatgtgaacgccattgcacagcaatatatagccgggcggaaagaccttcatgtaatcggcattgatcgcggtgagcggaatctgatctacgtgtccgttattgacatgtatggccgcatattggaacagaagtcatttaacctggtcgagcaggtgagcagtcaaggaaccaaacggtactatgattacaaggaaaaactccagaatcgcgaggaagagcgggacaaggctcgccagtcttggaaaactatcgggaagattaaagaacttaaggagggctatctgagctccgtaatccacgaaattgcccaaatggtggttaaatacaacgcaataatcgccatggaggatttgaattatggtttcaagcggggccgctttaaagtcgaacggcaggtgtaccagaagttcgagaccatgctgatttcaaaactcaactatcttgctgacaagagccaagccgtagatgaacccggagggattctgcgcggctaccagatgacatatgtgccggacaatattaaaaacgttggtcggcagtgcggcataatcttttacgtccctgcagcctataccagtaagattgatcccactaccggattcatcaatgcttttaaacgcgacgtggtatctacaaacgatgccaaggagaatttcttgatgaaatttgacagcattcaatacgatatagaaaaggggctgttcaaattttccttcgactataagaactttgcaacccataaactcactcttgccaagaccaaatgggatgtgtacacaaatggcacccggattcagaacatgaaggttgagggtcactggctgtcaatggaagtcgagttgactaccaaaatgaaggaactgctcgacgatagccatattccgtatgaggaaggccagaatatccttgacgatctgcgcgagatgaaagacattacaaccatagtgaacggaatcttggaaattttctggctgactgtacaactccggaatagtcgcatcgataacccagactacgatcggattatatctcccgtgcttaataagaacggggagtttttcgacagcgatgaatataattcctacatcgacgctcagaaagccccgctgcctattgatgcagacgccaacggcgctttttgtatcgccttgaagggtatgtataccgcaaatcagattaaagagaactgggttgaaggcgagaagctgcccgccgattgcctcaaaatagaacacgcttcatggcttgccttcatgcaaggagagcgcggg

SEQ ID NO: 9 - protein, 1307 aa, Artificial Sequence
  AsCas12a
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 10 - protein, 1228 aa, Artificial Sequence
  LbCas12a
AASKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGAAGYKSLFKKDIIETILPEAADDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNAKTKQALPKFKPLYKQVLSDRESLSFYGEGYTSDEEVLEVFRNTLNKNSEIFSSIKKLEKLFKNFDEYSSAGIFVKNGPAISTISKDIFGEWNLIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFAAAKKNNVFAWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 11 - protein, 1300 aa, Artificial Sequence
  FnCas12a
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 12 - RNA, 19 nt, Artificial Sequence
  5′-handle RNA
aauuucuacuguuguagau

SEQ ID NO: 13 - protein, 1263 aa, Artificial Sequence
  d_mgCas12a-1
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQAEKRKAIYKKFADDDRFKNMFSAKLISDILPEFVIHNNNYSASEKEEKTQVIKLFSRFATSFKDYFKNRANCFSADDISSSSCHRIVNDNAEIFFSNALVYRRIVKNLSNDDINKISGDIKDSLKEMSLEEIYSYEKYGEFITQEGISFYNDICGKVNSFMNLYCQKNKENKNLYKLRKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCPDDNIKAETYIHEISHILNNFEAQELKYNPEIHLVESELKASELKNVLDVIMNAFHWCSVFMTEELVDKDNNFYAELEEIYDEIYTVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHLKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKTIPENIYQELYKYFNDKSDKELSDEAAKLKNVVGHHEAATNIVKDYRYTYDKYFLHMPITINFKANKTSFINDRILQYIAKEKNLHVIGIARGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKKLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 14 - protein, 1275 aa, Artificial Sequence
  d_mgCas12a-2
MGKNQNFQEFIGVSPLQKTLRNELIPTETTKKNITQLDLLTEDEIRAQNREKLKEMMDDYYRNVIDSTLHVGIAVDWSYLFSCMRNHLRENSKESKRELERTQDSIRSQIHNKFAERADFKDMFGASIITKLLPTYIKQNSEYSERYDESMEILKLYGKFTTSLTDYFETRKNIFSKEKISSAVGYRIVEENAEIFLQNQNAYDRICKIAGLDLHGLDNEITAYVDGKTLKEVCSDEGFAKAITQEGIDRYNEAIGAVNQYMNLLCQKNKALKPGQFKMKRLHKQILCKGTTSFDIPKKFENDKQVYDAVNSFTEIVTKNNDLKRLLNITQNANDYDMNKIYVVADAYSMISQFISKKWNLIEECLLDYYSDNLPGKGNAKENKVKKAVKEETYRSVSQLNEVIEKYYVEKTGQSVWKVESYISSLAEMIKLELCHEIDNDEKHNLIEDDEKISEIKELLDMYMDVFHIIKVFRVNEVLNFDETFYSEMDEIYQDMQEIVPLYNHVRNYVTQKPYKQEKYRLYFHTPTLANGWSKSKEYDNNAIILVREDKYYLGILNAKKKPSKEIMAGKEDCSEHAYAKMNYYLLPGANKMLPKVFLSKKGIQDYHPSSYIVEGYNEKKHIKGSKNFDIRFCRDLIDYFKECIKKHPDWNKFNFEFSATETYEDISVFYREVEKQGYRVEWTYINSEDIQKLEEDGQLFLFQIYNKDFAVGSTGKPNLHTLYLKNLFSEENLRDIVLKLNGEAEIFFRKSSVQKPVIHKCGSILVNRTYEITESGTTRVQSIPESEYMELYRYFNSEKQIELSDEAKKYLDKVQCNKAKTDIVKDYRYTMDKFFIHLPITINFKVDKGNNVNAIAQQYIAGRKDLHVIGIARGERNLIYVSVIDMYGRILEQKSFNLVEQVSSQGTKRYYDYKEKLQNREEERDKARKSWKTIGKIKELKEGYLSSVIHEIAQMVVKYNAIIAMEDLNYGFKRGRFKVERQVYQKFETMLISKLNYLADKSQAVDEPGGILRGYQMTYVPDNIKNVGRQCGIIFYVPAAYTSKIDPTTGFINAFKRDVVSTNDAKENFLMKFDSIQYDIEKGLFKFSFDYKNFATHKLTLAKTKWDVYTNGTRIQNMKVEGHWLSMEVELTTKMKELLDDSHIPYEEGQNILDDLREMKDITTIVNGILEIFWLTVQLRNSRIDNPDYDRIISPVLNKNGEFFDSDEYNSYIDAQKAPLPIDADANGAFCIALKGMYTANQIKENWVEGEKLPADCLKIEHASWLAFMQGERG

SEQ ID NO: 15 - DNA, 53 bp, Artificial Sequence
  CCR5 Adapter primer sequence (5′-3′)
tcgtcggcagcgtcagatgtgtataagagacagggtatttctgttcagatcac

SEQ ID NO: 16 - DNA, 55 bp, Artificial Sequence
  CCR5 Adapter primer sequence (5′-3′)
gtctcgtgggctcggagatgtgtataagagacaggcccatcaattatagaaagcc

SEQ ID NO: 17 - DNA, 53 bp, Artificial Sequence
  DNMT1 Adapter primer sequence (5′-3′)
tcgtcggcagcgtcagatgtgtataagagacagctgcacacagcaggcctttg

SEQ ID NO: 18 - DNA, 54 bp, Artificial Sequence
  DNMT1 Adapter primer sequence (5′-3′)
gtctcgtgggctcggagatgtgtataagagacagcccaataagtggcagagtgc

SEQ ID NO: 19 - RNA, 28 nt, Artificial Sequence
  NbFTa14_1/2-2 crRNA sequence(PAM site)
uuuggauaauuuguacucuugucgaugu

SEQ ID NO: 20 - RNA, 28 nt, Artificial Sequence
  NbFTa14_1/2-4 crRNA sequence(PAM site)
uuuaguccacaaacagcuaagcccacau

SEQ ID NO: 21 - DNA, 20 bp, Artificial Sequence
  NGS NbFTa14_1 Forward primer
tgagctgaagatggattatg

SEQ ID NO: 22 - DNA, 20 bp, Artificial Sequence
  NGS NbFTa14_1 Reverse primer
tcatgcttaagataaaagag

SEQ ID NO: 23 - DNA, 20 bp, Artificial Sequence
  NGS NbFTa14_2 Forward primer
tcatgagcttaagatggatc

SEQ ID NO: 24 - DNA, 20 bp, Artificial Sequence
  NGS NbFTa14_2 Reverse primer
gtttaagctaaaagaactac

SEQ ID NO: 25 - RNA, 43 nt, Artificial Sequence
  LsXTb12 crRNA #1
aauuucuacuaaguguagauucuucauccucaauuccaucacc

SEQ ID NO: 26 - RNA, 43 nt, Artificial Sequence
  LsXTb12 crRNA #2
aauuucuacuaaguguagaugcaagccuguaacucuggaagac

SEQ ID NO: 27 - DNA, 1504 bp, Artificial Sequence
  HsCCR5 Linear DNA
ggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctg

SEQ ID NO: 28 - DNA, 1119 bp, Artificial Sequence
  HsDNMT1 Linear DNA
gctgctctcgaactcctggcctcaactaatccacctgccttggcctcccaaagtgctgggattacaggcgtgagccactgctcccagccccacgtgtctttgtctcaagtctttctgaagctcttcaaaggcccagtgacttgtggctgtggggcgggatgatgggccagttggagggtccaaggatcttgtgctggaagggttttgggcccatgtgagcaggaccagaacccttccccaaggggtgcaatgcccaggttgtcctccatctgagcaggggctggcagtacacctgcccccgggccttgggcctgggtgtccacatcaggcattgcccttctcccctcctgcaggtgggcaatgccgtgccaccgcccctggccaaagccattggcttggagatcaagctttgtatgttggccaaagcccgagagagtgcctcaggtatggtggggtgggccaggcttcctctggggcctgactgccctctgggggtacatgtgggggcagttgctggccaccgttttgggctctgggactcaggcgggtcacctacccacgttcgtggccccatctttctcaaggggctgctgtgaggattgagtgagttgcacgtgtcaagtgcttagagcaggcgtgctgcacacagcaggcctttggtcaggttggctgctgggctggccctggggccgtttccctcactcctgctcggtgaatttggctcagcaggcacctgcctcagctgctcacttgagcctctgggtctagaaccctctggggaccgtttgaggagtgttcagtctccgtgaacgttcccttagcactctgccacttattgggtcagctgttaacatcagtacgttaatgtttcctgatggtccatgtctgttactcgcctgtcaagtggcgtgacaccgggcgtgttccccagagtgacttttccttttatttcccttcagctaaaataaaggaggaggaagctgctaaggactagttctgccctcccgtcacccctgtttctggcaccaggaatccccaacatgcactgatgttgtgtttttaacatgtcaatctgtccgttcacatgtgtggtacatggtgtttgtggccttggctgac

SEQ ID NO: 29 - DNA, 1460 bp, Artificial Sequence
  HsEMX1 Linear DNA
gtggggacagaaggtctggagctgcccgtgaagggcagaatgctgccctcagacccgcttcctccctgtccttgtctgtccaaggagaatgaggtctcactggtggatttcggactaccctgaggagctggcacctgagggacaaggccccccacctgcccagctccagcctctgatgaggggtgggagagagctacatgaggttgctaagaaagcctcccctgaaggagaccacacagtgtgtgaggttggagtctctagcagcgggttctgtgcccccagggatagtctggctgtccaggcactgctcttgatataaacaccacctcctagttatgaaaccatgcccattctgcctctctgtatggaaaagagcatggggctggcccgtggggtggtgtccactttaggccctgtgggagatcatgggaacccacgcagtgggtcataggctctctcatttactactcacatccactctgtgaagaagcgattatgatctctcctctagaaactcgtagagtcccatgtctgccggcttccagagcctgcactcctccaccttggcttggctttgctggggctagaggagctaggatgcacagcagctctgtgaccctttgtttgagaggaacaggaaaaccacccttctctctggcccactgtgtcctcttcctgccctgccatccccttctgtgaatgttagacccatgggagcagctggtcagaggggaccccggcctggggcccctaaccctatgtagcctcagtcttcccatcaggctctcagctcagcctgagtgttgaggccccagtggctgctctgggggcctcctgagtttctcatctgtgcccctccctccctggcccaggtgaaggtgtggttccagaaccggaggacaaagtacaaacggcagaagctggaggaggaagggcctgagtccgagcagaagaagaagggctcccatcacatcaaccggtggcgcattgccacgaagcaggccaatggggaggacatcgatgtcacctccaatgactagggtgggcaaccacaaacccacgagggcagagtgctgcttgctgctggccaggcccctgcgtgggcccaagctggactctggccactccctggccaggctttggggaggcctggagtcatggccccacagggcttgaagcccggggccgccattgacagagggacaagcaatgggctggctgaggcctgggaccacttggccttctcctcggagagcctgcctgcctgggcgggcccgcccgccaccgcagcctcccagctgctctccgtgtctccaatctcccttttgttttgatgcatttctgttttaatttattttccaggcaccactgtagtttagtgatccccagtgtcccccttccctatgggaataataaaagtctctctcttaatgacacgggcatccagctccagccccaga

SEQ ID NO: 30 - DNA, 5311 bp, Artificial Sequence
  All-in-one vector(HsCCR5)
cttccgcttcctcgctcactgattcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgaatccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctggctccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgccccggcgtgtcaataatatcactctgtacatccacaaacagacgataacggctctctcttttataggtgtaaaccttaaactgccgtacgtataggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgattcactatagggcgaattgggccctctagatgcatgctcgagcggccgccagtgtgatggatatctgcagaattcgcccttggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgaagggcgaattccagcacactggcggccgttactagtggatccgagctcggtaccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgct

SEQ ID NO: 31 - DNA, 4926 bp, Artificial Sequence
  All-in-one vector(HsDNMT1)
cttccgcttcctcgctcactgattcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgaatccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctggctccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgccccggcgtgtcaataatatcactctgtacatccacaaacagacgataacggctctctcttttataggtgtaaaccttaaactgccgtacgtataggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgattcactatagggcgaattgggccctctagatgcatgctcgagcggccgccagtgtgatggatatctgcagaattcgcccttgctgctctcgaactcctggcctcaactaatccacctgccttggcctcccaaagtgctgggattacaggcgtgagccactgctcccagccccacgtgtctttgtctcaagtctttctgaagctcttcaaaggcccagtgacttgtggctgtggggcgggatgatgggccagttggagggtccaaggatcttgtgctggaagggttttgggcccatgtgagcaggaccagaacccttccccaaggggtgcaatgcccaggttgtcctccatctgagcaggggctggcagtacacctgcccccgggccttgggcctgggtgtccacatcaggcattgcccttctcccctcctgcaggtgggcaatgccgtgccaccgcccctggccaaagccattggcttggagatcaagctttgtatgttggccaaagcccgagagagtgcctcaggtatggtggggtgggccaggcttcctctggggcctgactgccctctgggggtacatgtgggggcagttgctggccaccgttttgggctctgggactcaggcgggtcacctacccacgttcgtggccccatctttctcaaggggctgctgtgaggattgagtgagttgcacgtgtcaagtgcttagagcaggcgtgctgcacacagcaggcctttggtcaggttggctgctgggctggccctggggccgtttccctcactcctgctcggtgaatttggctcagcaggcacctgcctcagctgctcacttgagcctctgggtctagaaccctctggggaccgtttgaggagtgttcagtctccgtgaacgttcccttagcactctgccacttattgggtcagctgttaacatcagtacgttaatgtttcctgatggtccatgtctgttactcgcctgtcaagtggcgtgacaccgggcgtgttccccagagtgacttttccttttatttcccttcagctaaaataaaggaggaggaagctgctaaggactagttctgccctcccgtcacccctgtttctggcaccaggaatccccaacatgcactgatgttgtgtttttaacatgtcaatctgtccgttcacatgtgtggtacatggtgtttgtggccttggctgacaagggcgaattccagcacactggcggccgttactagtggatccgagctcggtaccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgct

SEQ ID NO: 32 - DNA, 5267 bp, Artificial Sequence
  All-in-one vector(HsEMX1)
cttccgcttcctcgctcactgattcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgaatccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctggctccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgccccggcgtgtcaataatatcactctgtacatccacaaacagacgataacggctctctcttttataggtgtaaaccttaaactgccgtacgtataggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgattcactatagggcgaattgggccctctagatgcatgctcgagcggccgccagtgtgatggatatctgcagaattcgcccttgtggggacagaaggtctggagctgcccgtgaagggcagaatgctgccctcagacccgcttcctccctgtccttgtctgtccaaggagaatgaggtctcactggtggatttcggactaccctgaggagctggcacctgagggacaaggccccccacctgcccagctccagcctctgatgaggggtgggagagagctacatgaggttgctaagaaagcctcccctgaaggagaccacacagtgtgtgaggttggagtctctagcagcgggttctgtgcccccagggatagtctggctgtccaggcactgctcttgatataaacaccacctcctagttatgaaaccatgcccattctgcctctctgtatggaaaagagcatggggctggcccgtggggtggtgtccactttaggccctgtgggagatcatgggaacccacgcagtgggtcataggctctctcatttactactcacatccactctgtgaagaagcgattatgatctctcctctagaaactcgtagagtcccatgtctgccggcttccagagcctgcactcctccaccttggcttggctttgctggggctagaggagctaggatgcacagcagctctgtgaccctttgtttgagaggaacaggaaaaccacccttctctctggcccactgtgtcctcttcctgccctgccatccccttctgtgaatgttagacccatgggagcagctggtcagaggggaccccggcctggggcccctaaccctatgtagcctcagtcttcccatcaggctctcagctcagcctgagtgttgaggccccagtggctgctctgggggcctcctgagtttctcatctgtgcccctccctccctggcccaggtgaaggtgtggttccagaaccggaggacaaagtacaaacggcagaagctggaggaggaagggcctgagtccgagcagaagaagaagggctcccatcacatcaaccggtggcgcattgccacgaagcaggccaatggggaggacatcgatgtcacctccaatgactagggtgggcaaccacaaacccacgagggcagagtgctgcttgctgctggccaggcccctgcgtgggcccaagctggactctggccactccctggccaggctttggggaggcctggagtcatggccccacagggcttgaagcccggggccgccattgacagagggacaagcaatgggctggctgaggcctgggaccacttggccttctcctcggagagcctgcctgcctgggcgggcccgcccgccaccgcagcctcccagctgctctccgtgtctccaatctcccttttgttttgatgcatttctgttttaatttattttccaggcaccactgtagtttagtgatccccagtgtcccccttccctatgggaataataaaagtctctctcttaatgacacgggcatccagctccagccccagaaagggcgaattccagcacactggcggccgttactagtggatccgagctcggtaccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgct